SEQUENCE LISTING (11 sequences)

SEQ ID NO: 1 - protein, 4 aa, synthetic construct
  REGION 1..4: note = peptide
HDEL

SEQ ID NO: 2 - protein, 4 aa, synthetic construct
  REGION 1..4: note = peptide
KDEL

SEQ ID NO: 3 - protein, 21 aa, synthetic construct
  REGION 1..21: note = tail peptide
KLKHTKKRQIYTDIEMNRLGK

SEQ ID NO: 4 - protein, 6 aa, synthetic construct
  REGION 1..6: note = tail peptide
YTDIEM

SEQ ID NO: 5 - protein, 19 aa, synthetic construct
  REGION 1..19: note = C-terminal tail peptide
KFDEDDSEPVLKGVKLHYT

SEQ ID NO: 6 - protein, 21 aa, synthetic construct
  REGION 1..21: note = peptide
KLKHTKKRQIATDIEMNRLGK

SEQ ID NO: 7 - protein, 37 aa, synthetic construct
  REGION 1..37: note = peptide
PNKGSGTTSGTTRLLSGHTCFTLTGLLGTLVTMGLLT

SEQ ID NO: 8 - protein, 14 aa, synthetic construct
  REGION 1..14: note = C-terminal tail peptide
DSEPVLKGVKLHYT

SEQ ID NO: 9 - protein, 38 aa, synthetic construct
  REGION 1..38: note = transmembrane domain and tail
SGKLYLIGIAAIVVAVIMIFEMILSMVLCCGIRNSSVY

SEQ ID NO: 10 - protein, 21 aa, synthetic construct
  REGION 1..21: note = tail peptide
KLKHTKKRQIAAAAAANRLGK

SEQ ID NO: 11 - protein, 6 aa, synthetic construct
  REGION 1..6: note = tail peptide
RNSSVY

(19) United States
(12) Patent Application Publication
(10) Pub. No.: US 2024/0336660 A1
(43) Pub. Date: Oct. 10, 2024

(54) METHOD FOR SURFACE EXPRESSION OF MEMBRANE PROTEINS THAT HAVE A CYTOPLASMIC C-TERMINAL TAIL

(71) Applicants: The Johns Hopkins University, Baltimore, MD (US); Capricor, Inc., Beverly Hills, CA (US)

(72) Inventors: Stephen J. Gould, Baltimore, MD (US); Chenxu Guo, Baltimore, MD (US); Shang-Jui Tsai, Baltimore, MD (US); Linda Marban, Beverly Hills, CA (US)

(21) Appl. No.: 18/577,642

(22) PCT Filed: Jul. 8, 2022
(86) PCT No.: PCT/US2022/036553

§ 371 Date: Jan. 8, 2024

Related U.S. Application Data
(60) Provisional application No. 63/220,350, filed on Jul. 9, 2021.

(51) Int. Cl. C07K 14/005; A61K 9/50; A61K 39/00

(52) U.S. Cl. CPC C07K 14/005; A61K 9/5068; A61K 39/00; C07K 2319/02; C07K 2319/03; C12N 2770/20022; C12N 2770/20034

(57) ABSTRACT

Coronavirus egress is mediated by lysosomal exocytosis. It is demonstrated herein that the D614G mutation enhances Spike trafficking to lysosomes and the lysosomal accumulation of newly synthesized virus particles, augments Spike-mediated disruption of endomembrane homeostasis, and causes a 3-fold reduction in cell surface Spike expression. Moreover, it is shown that the D614G mutation is an intragenic suppressor of the 12 nucleotide-long furin cleavage site (FCS) insertion, restoring Spike trafficking to lysosomes and TMPRSS2-independent infectivity, both of which had been impaired by the prior FCS insertion mutation. This data identifies enhanced lysosomal sorting as the earliest known manifestation of the D614G mutation, have implications for virus evolution, immunity, and vaccine design, and support a lysosomal model of coronavirus biogenesis and entry.

### CROSS-REFERENCE TO RELATED APPLICATIONS

This application claims benefit of priority under 35 U.S.C. § 119(e) of U.S. Provisional Patent Application No. 63/220,350, filed Jul. 9, 2021. The disclosure of the prior application is considered part of and is incorporated by reference in the disclosure of this application.

### BACKGROUND OF THE INVENTION

### Field of the Invention

The present invention relates generally to methods of increasing the expression of membrane proteins in the surface of cells, exosomes and other extracellular vesicles, more specifically to methods of enhancing immunogenicity and efficacy of proteins encoded by an expression dependent vaccine.

### Background Information

COVID-19 (coronavirus infectious disease 2019) is caused by the enveloped virus SARS CoV-2. Coronaviruses have the largest genomes of all RNA viruses, and SARS-CoV-2 encodes more than two dozen proteins, most of which are essential for virus replication. Of these, the Spike protein is particularly critical because it mediates virus-cell binding, catalyzes fusion of viral and cellular membranes, is the sole antigen expressed by most expression dependent SARS-CoV-2 vaccines and is the primary target of most antibody-based therapeutics.

Several lines of evidence indicate that coronavirus assembly and budding occurs in the ER-Golgi intermediate compartment (ERGIC). Newly-synthesized coronavirus particles are then trafficked to lysosomes, where they accumulate until they are released “en masse” by an Arl8-dependent pathway of lysosomal exocytosis. Not surprisingly, coronavirus infection leads to a reprogramming of host endomembrane systems, allowing the virus to convert lysosomes from degradative compartments to biogenic organelles that store newly-synthesized virus particles and mediate their subsequent release. This involves a number of discrete and measurable phenotypes, including lysosome deacidification, lysosome clustering, lysosomal accumulation of 4 KDEL receptors, disruption of ER protein homeostasis, and impaired lysosomal uptake of endocytosed materials.

The lysosomal pathway of coronavirus egress mirrors the lysosomal pathway of coronavirus entry. This pathway is characterized by the rapid appearance of coronavirus particles in lysosomes shortly after receptor binding, as well as essential roles for lysosomal proteases, the vacuolar ATPase, and lysosomal trafficking factors in the early stages of coronavirus infection.

Prior to the COVID-19 pandemic, an ancestor of SARS-CoV-2 acquired a 12 nucleotide (nt)-long insertion mutation that inserted 4 amino acids into the Spike protein near its S1/S2 processing site (681PRRA684), creating a furin cleavage site (FCS) where none had previously existed (681PRRAR|S686). This FCS insertion mutation dramatically altered Spike protein structure and conferred a number of advantageous traits, including furin-mediated cleavage in the virus-producing cell, dramatically enhanced infectivity via a serine protease (TMPRSS2 or equivalent)-dependent pathway, enhanced infectivity of airway epithelial cells in vitro, acquisition of a second receptor that broadens its host cell tropism (neuropilin-1), and superior transmission in vivo. These 5 traits underscore the critical importance of this mutation to the evolution of SARS-CoV-2 into a human pathogen. Furthermore, the fact that the FCS insertion mutation is absent from its closest viral relatives indicates that this mutation arose in a near-immediate ancestor of SARS-CoV-2.

Shortly after its zoonotic leap to humans, SARS-CoV-2 acquired yet another strongly advantageous mutation in its Spike gene, D614G. This mutation confers a pronounced increase in SARS-CoV-2 infectivity by both the TMPRSS2-dependent and TMPRSS2-independent pathways, as well as increased viral load and superior transmission. There is now abundant evidence that the D614G mutation causes a significant increase in the early phases of SARS-CoV-2 entry. As for whether these D614G-mediated phenotype are the result of D614G-mediated changes in affinity for its receptor angiotensin converting enzyme-2 (ACE2), current evidence is mixed, with some studies concluding that the D614G mutation mediates a subtle decrease in D614G-ACE2 binding kinetics while others suggest it causes a subtle increase. Into this rich array of empirical observations, we add the observations that the D614G mutation enhances Spike trafficking to lysosomes, augments a heretofore unrecognized role for Spike in disruption of endomembrane homeostasis, reduces cell surface Spike expression, and suppresses multiple deleterious traits associated with the FCS insertion mutation.

### SUMMARY OF THE INVENTION

The present invention is based on the seminal discovery that the simultaneous engineering of two general changes to the protein of interest, removal of retrieval signals and addition of ER export signals increase the expression of membrane proteins in the surface of cells, exosomes and other extracellular vesicles.

In one embodiment, the present invention provides an isolated non-naturally occurring cell-surface protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence. In one aspect, the protein is a therapeutic protein or an antigenic protein.

In another aspect, the carboxy-terminal cytoplasmic domain has an endoplasmic reticulum (ER) export signal. In certain aspects, the cytoplasmic domain does not have an HDEL (SEQ ID NO:1) or a KDEL (SEQ ID NO:2) sequence. In an additional aspect, the protein has the sequence KLKHTKKRQIYTDIEMNRLGK (SEQ ID NO:3) at the carboxy-terminus of the protein. In a further aspect, the protein has the sequence Tyr19-Thr20-Asp21-Ile22-Glu23-Met24 (SEQ ID NO:4) of vesicular stomatitis virus glycoprotein (VSV G) tail at the carboxy-terminus.

In one aspect, the protein is an antigenic protein from a eukaryote, a prokaryote or a virus. In certain aspects, the virus is a coronavirus. In specific aspects, the coronavirus is SARS-CoV-2. In another aspect, the antigenic protein is spike protein. In an additional aspect, all or part of the sequence of the cytoplasmic domain of the SARS-CoV-2 spike protein is removed. In a further aspect, the sequence KFDEDDSEPVLKGVKLHYT_{COOH} (SEQ ID NO:5) in the cytoplasmic domain of the spike protein is removed. In certain aspects, the SARS-CoV-2 spike protein is the Wuhan-1 strain SARS-CoV-2 spike protein; a furin-blocked, trimer-stabilized form of the Wuhan-1 strain SARS-CoV-2 spike protein; the Wuhan-1 strain SARS-CoV-2 spike protein with an amino acid change of D614G; the Wuhan-1 strain SARS-CoV-2 spike protein with di-proline substitutions of 986KV987-to-986PP987 (S-2P); the Wuhan-1 strain SARS-CoV-2 spike protein with cleavage site mutations of 682RRAR685-to-682GSAG685, or equivalent (S-CSM); or the Wuhan-1 strain SARS-CoV-2 spike protein with both S-2P and S-CSM mutations.

In an additional embodiment, the present invention provides, an isolated nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence. In one aspect, the recombinant nucleic acid molecule is DNA, RNA, or messenger RNA (mRNA). In an additional aspect, the nucleic acid further has an expression control sequence operatively linked to the nucleic acid sequence. In a further aspect, the nucleic acid sequence is in a plasmid or a viral vector.

In a further embodiment, the present invention provides an isolated cell with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence. In one aspect, the cell is a mammalian cell. In certain aspects, the mammalian cell is a human cell.

In one embodiment, the present invention provides a delivery vehicle with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence. In one aspect, the nucleic acid delivery vehicle is an extracellular vesicle (EV), a lipid nanoparticle (LNP), a liposome, a plasmid, or a viral vector. In an aspect, the EV is an exosome or a microvesicle. In certain aspects, the viral vector is an adenoviral vector, an adeno-associated vector (AAV), or a lentiviral vector. In a specific aspect, the delivery vehicle is an EV. In an additional aspect, the engineered antigenic protein is configured to elicit a humoral immune response and/or a cellular immune response in an animal subject. In certain aspects, the animal subject is a human subject.

In an additional embodiment, the present invention provides a pharmaceutical composition having a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence and a physiologically acceptable excipient and/or adjuvant.

In a further aspect, the present invention provides a pharmaceutical composition having a delivery vehicle with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence and a physiologically acceptable excipient and/or adjuvant. In one aspect, the delivery vehicle has an EV and a chemical lipofection reagent or a chemical transfection reagent. In a specific aspect, the EV is an exosome or a microvesicle. In an additional aspect, the chemical lipofection reagent or the chemical transfection reagent is a polycationic lipid. In a further aspect, the polycationic lipid is an mRNA lipofection reagent or an mRNA transfection reagent.

In one embodiment, the present invention provides a method of making a nucleic acid delivery vehicle by loading a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence into an extracellular vesicle (EV). In one aspect, the EV is an exosome or a microvesicle. In an additional aspect, the nucleic acid molecules are pre-mixed with a chemical lipofection reagent or a chemical transfection reagent. In certain aspects, the chemical lipofection reagent or the chemical transfection reagent is a polycationic lipid. In some aspects, the polycationic lipid is an mRNA lipofection reagent or an mRNA transfection reagent.

In an additional embodiment, the present invention provides a method comprising administering into an animal a pharmaceutical composition having a delivery vehicle with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence and a physiologically acceptable excipient and/or adjuvant. In one aspect, the administration elicits an antigenic immune response in a subject. In an additional aspect, the animal is a mammal. In a specific aspect, the mammal is a human. In a further aspect, the antigen specific immune response comprises a T cell response and/or a B cell response. In one aspect, the administration is a single administration. In an additional aspect, there is a booster dose.

### BRIEF DESCRIPTION OF THE DRAWINGS

FIG. 1. COVID-19 patient plasmas identify Spike in a unique compartment. Immunoblot analysis of equal amounts of doxycycline-induced Htet1/SW1 and Htet1/SD614G 531 cell lysates, using a monoclonal antibody that binds Spike in the S1 domain (MG44) and a polyclonal antibody that recognizes endogenously expressed HSP90. MW markers 250 kDa, 150 kDa, 100 kDa, 75 kDa, 50 kDa, 37 kDa, 25 kDa, 20 kDa, 15 kDa, 10 kDa.

FIGS. 2A-2D. Spike is a lysosomal membrane protein. Electron micrographs of doxycycline-induced Htet1/SD614G cells (2 days) processed for immunogold electron microscopy using (A, B) rabbit anti-Spike antibodies and 12 nm gold conjugates of anti-rabbit secondary 27 antibodies, and (C, D) rabbit anti-Spike antibodies, mouse monoclonal antibody specific for Lamp2, 12 nm gold conjugates of anti-rabbit secondary antibodies, and 6 nm gold conjugates of anti-mouse secondary antibodies. Black arrowheads denote the positions of 12 nm gold directed against Spike and white arrowheads denote the positions of 6 nm gold directed against Lamp2. Bar, 200 nm.

FIGS. 3A and 3B. The D614G mutation directs Spike to lysosomes. (A) Immunoblot analysis of cell lysates collected from doxycycline-induced Htet1, Htet1/SW1 and Htet1/SD614G cells probed with (left panel) affinity-purified anti-Spike antibody specific for the C-terminal 14 amino acids of Spike and (right panel) a monoclonal antibody specific for the cellular protein Hsp90. MW size markers, from top: 250 kDa, 150 kDa, 100 kDa, 75 kDa, 50 kDa, 37 kDa, 25 kDa, 20 kDa, 15 kDa, 10 kDa. (B) Violin plot showing the amount of lysosome-localized Spike fluorescence, quantified from >30 immunofluorescence micrographs of doxycycline-induced Htet1/SW1 cells and Htet1/SD614G cells, following staining with the S.C14 affinity purified antibody and a monoclonal antibody to Lamp2. Large dotted line denotes the median, smaller dotted lines defining the middle 50% of samples, and **** denotes Student's t-test p value (2.4×10-7).

FIG. 4. Spike trafficking to lysosomes is mediated by its extracellular domain. Line diagram of Spike proteins carrying various permutations of its tail peptide and transmembrane domain.

FIGS. 5A-5C. Spike-induced, D614G-augmented disruption of endomembrane homeostasis. (A-C) Immunoblot of (left panels) secreted protein fractions and (right panels) cell-associated protein lysates, interrogated using antibodies specific for (A) calreticulin, (B) BiP/GRP78, and (C) beta-glucocerebrosidase. MW size markers, from top: 250 kDa, 150 kDa, 100 kDa, 75 kDa, 50 kDa, 37 kDa, 25 kDa, 20 kDa, 15 kDa, 10 kDa.

FIGS. 6A-6J. D614G suppresses the Spike trafficking defect caused by furin cleavage site insertion. (A-C) Flow cytometry of live, chilled, doxycycline-induced Htet1, Htet1/SW1 and Htet1/SD614G cells, labeled with Alexa Fluor 647-conjugate of 1A9 anti-S2 antibody. (D) Immunoblot of cell lysates prepared from doxycycline-induced Htet1, Htet1/SW1 and Htet1/SD614G cells, probed with the 1A9 anti-S2 antibody. MW size markers, from top: 250 kDa, 150 kDa, 100 kDa, 75 kDa, 50 kDa, 37 kDa, 25 kDa, 20 kDa, 15 kDa, 10 kDa. (E) Integrated Spike-reactive fluorescence values (arbitrary units) following flow cytometry of doxycycline-induced Htet1 and Htet1 derivative cell lines. (F) Flow cytometry of live, chilled, doxycycline-induced Htet1/SW1-CSM cells, labeled with Alexa Fluor 647-conjugate of 1A9 anti-S2 antibody. (G-H) Fluorescence micrographs of doxycycline induced (G) Htet1/SW1-CSM, (H) Htet1/SW1, and (I) Htet1/SD614G cells, stained with plasma G4 to detect Spike and Lamp2 to detect lysosomes. Bar, 50 μm. (J, K) Flow cytometry of live, chilled, doxycycline-induced (J) Htet1/SW1-2P cells and (K) Htet1/SW1-30 2P-CSM cells, labeled with Alexa Fluor 647-conjugate of 1A9 anti-S2 antibody. (L) Immunoblot analysis of equal amounts of doxycycline-induced Htet1, Htet1/SW1, Htet1/SD614G, Htet1/SW1-2P, Htet1/SW1-CSM, and Htet1/SW1-2P-CSM cell lysates, using a monoclonal antibody that binds Spike in the S1 domain (MG44). (M) Line diagram of Spike proteins corresponding to Htet1, Htet1/SW1, Htet1/SD614G, Htet1/SW1-2P, Htet1/SW1 CSM, and Htet1/SW1 619-2P-CSM.

FIGS. 7A-7E. Inhibition of furin cleavage reduces cell surface Spike expression. (A-D) Anti Spike flow cytometry of doxycycline-induced Htet1 cells, Htet1/SW1 cells, and Htet1/SW1 cells incubated with the furin cleavage inhibitor CMK. (E) Immunoblot of cell lysates of from doxycycline-induced Htet1 cells, Htet1/SW1 cells, and Htet1/SW1 cells incubated with the furin cleavage inhibitor CMK using antibodies specific to (left panel) the C-terminal 14 amino acids of Spike (S.C14) and (right panel) HSP90.

FIGS. 8A-8G. Combining 2P, tail deletion and ER export signal maximizes surface expression of SD614G. (A-E) Flow cytometry of live, chilled, (A) Htet1 cells or (B-E) Htet1 cells that had been transfected two days earlier with equal amounts of DNA of CMV based expression vectors designed to express (B) SD614G, (C) SD614G-2P, (D) SD614G-ECD2P-GPI, or (E) SD614G-DC-2P-VTYA. (F) Integrated Spike-reactive fluorescence values (arbitrary units) following flow cytometry of Htet1 and transfected Htet1 cell populations. Immunoblot of cell lysates prepared from Htet1 cells that had been transfected two days earlier with equal amounts of DNA of CMV-based expression vectors designed to express SD614G, SD614G-2P, SD614G-ECD-2P-GPI, or SD614G 840-DC-2P-VTYA. MW size markers, from top: 250 kDa, 150 kDa, 100 kDa, 75 kDa, 50 kDa, 37 kDa, 25 kDa, 20 kDa, 15 kDa, 10 kDa. (G) Line diagram of Spike proteins corresponding to SD614G, SD614G-2P, SD614G-ECD-2P-GPI, and SD614G-DC-2P-VTYA.

FIG. 9. A lysosomal model of coronavirus egress and entry. Schematic of (left panel) the lysosomal pathway of Spike and virus particle trafficking in virus-producing cells and (right panel) a lysosomal, TMPRSS2-independent pathway of SARS-CoV-2 entry.

### DETAILED DESCRIPTION OF THE INVENTION

The present invention is based on the seminal discovery that the simultaneous engineering of two general changes to the protein of interest, removal of retrieval signals and addition of ER export signals increase the expression of membrane proteins in the surface of cells, exosomes and other extracellular vesicles.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides an isolated non-naturally occurring cell-surface protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence. In one aspect, the protein is a therapeutic protein or an antigenic protein.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence. Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

The cell surface protein described herein are modified to have “improved cell surface expression”. By improved cell surface expression, it is meant that the modification of the protein results in a greater amount of protein being expressed at the surface of the cell, a extended half-life of the protein resulting in extended expression at the surface of the cell, or a combination of both.

An “antigen” according to the invention covers any substance that elicits an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, the antigen is a protein, also referred to as an “antigenic protein”. In the context of the embodiments of the present invention, the antigen is preferably presented by a cell, preferably by an antigen presenting cell which includes a diseased cell, in particular a infected cell, in the context of MHC molecules, which results in an immune reaction against the antigen. An antigen is preferably a product which corresponds to or is derived from a naturally occurring antigen. Such naturally occurring antigens include viral antigens. In another aspect, the carboxy-terminal cytoplasmic domain has an endoplasmic reticulum (ER) export signal. In certain aspects, the cytoplasmic domain does not have an HDEL (SEQ ID NO:1) or a KDEL (SEQ ID NO:2) sequence. In an additional aspect, the protein has the sequence KLKHTKKRQIYTDIEMNRLGK (SEQ ID NO:3) at the carboxy-terminus of the protein. In a further aspect, the protein has the sequence Tyr19-Thr20-Asp21-Ile22-Glu23-Met24 (SEQ ID NO:4) of vesicular stomatitis virus glycoprotein (VSV G) tail at the carboxy-terminus.

In one aspect, the protein is an antigenic protein from a eukaryote, a prokaryote or a virus.

In certain aspects, the virus is a coronavirus.

Coronaviruses are a group of related RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS and COVID-19, which is causing an ongoing pandemic. In cows and pigs, they cause diarrhea, while in mice they cause hepatitis and encephalomyelitis. Coronaviruses constitute the subfamily Orthocoronavirinae, in the family Coronaviridae, order Nidovirales and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

In specific aspects, the coronavirus is SARS-CoV-2.

In another aspect, the antigenic protein is spike protein. In an additional aspect, all or part of the sequence of the cytoplasmic domain of the SARS-CoV-2 spike protein is removed. In a further aspect, the sequence KFDEDDSEPVLKGVKLHYT_{COOH} (SEQ ID NO:5) in the cytoplasmic domain of the spike protein is removed. In certain aspects, the SARS-CoV-2 spike protein is the Wuhan-1 strain SARS-CoV-2 spike protein; a furin-blocked, trimer-stabilized form of the Wuhan-1 strain SARS-CoV-2 spike protein; the Wuhan-1 strain SARS-CoV-2 spike protein with an amino acid change of D614G; the Wuhan-1 strain SARS-CoV-2 spike protein with di-proline substitutions of 986KV987-to-986PP987 (S-2P); the Wuhan-1 strain SARS-CoV-2 spike protein with cleavage site mutations of 682RRAR685-to-682GSAG685, or equivalent (S-CSM); or the Wuhan-1 strain SARS-CoV-2 spike protein with both S-2P and S-CSM mutations.

In an additional embodiment, the present invention provides, an isolated nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence.

As used herein, the term “nucleic acid” or“oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e., transfection of, cells, in particular, in the form of RNA which can be prepared by in vitro transcription from a DNA template. The RNA can moreover be modified before application by stabilizing sequences, capping, and polyadenylation.

The term “sample” may include whole blood, plasma, serum, buffy coat, a body fluid, lymphocytes, tissue, amniotic fluid, cultured cells and the like. The term “sample” may also refer to a urine sample, saliva sample, blood cell-free DNA and specimen of or from the skin, mucous membrane or other body area or surface to be examined by means of a swab. In various embodiments, the mean use to collect the sample may contain a preservative. The preservative may include preservatives as hydrochloric acid, boric acid, acetic acid, toluene or thymol.

The nucleic acid may be extracted from the sample, by any method known in the art including by using organic solvents such as a mixture of phenol and chloroform, followed by precipitation with ethanol. Among other methods of extracting cell-free nucleic acid, one such method includes, for example, using polylysine-coated silica particles. Alternatively, the cell-free DNA may be extracted using commercially available kit such as, for example, QIAamp® DNA minikit (Qiagen, Germantown, MD).

The extracted nucleic acid is amplified by one of the alternative methods for amplification well known in the art, which include for example: the Xmap® technology of Luminex that allows the simultaneous analysis of up to 500 bioassays through the reading of biological test on the surface of microscopic polystyrene bead; the multiplex PCR that allows the simultaneous amplification of several DNA sequences; the multiplex ligation-dependent probe amplification (MLPA) for the amplification of multiple targets using a single pair of primers; the quantitative PCR (qPCR), which measures and quantify the amplification in real time; the ligation chain reaction (LCR) that uses primers covering the entire sequence to amplify, thereby preventing the amplification of sequences with a mutation; the rolling circle amplification (RCA), wherein the two ends of the sequences are joined by a ligase prior to the amplification of the circular DNA; the helicase dependent amplification (HDA) which relies on a helicase for the separation of the double stranded DNA; the loop mediated isothermal amplification (LAMP) which employs a DNA polymerase with high strand displacement activity; the nucleic acid sequence based amplification, specifically designed for RNA targets; the strand displacement amplification (SDA) which relies on a strand-displacing DNA polymerase, to initiate replication at nicks created by a strand-limited restriction endonuclease or nicking enzyme at a site contained in a primer; and the multiple displacement amplification (MDA), based on the use of the highly processive and strand displacing DNA polymerase from the bacteriophage Ø29. amplification methods as used herein have been used and tested, and are well known in the art.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, that relies agarose or polyacrylamide gel or High-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663.

Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

In one aspect, the recombinant nucleic acid molecule is DNA, RNA, or messenger RNA (mRNA).

The term “recombinant” in the context of the present invention means “made through genetic engineering”. Preferably, a “recombinant entity” such as a recombinant polypeptide in the context of the present invention is not occurring naturally, and preferably is a result of a combination of entities such as amino acid or nucleic acid sequences which are not combined in nature. For example, a recombinant polypeptide in the context of the present invention may contain several amino acid sequences derived from different proteins or different portions of the same protein fused together, e.g., by peptide bonds or appropriate linkers.

In an additional aspect, the nucleic acid further has an expression control sequence operatively linked to the nucleic acid sequence. In a further aspect, the nucleic acid sequence is in a plasmid or a viral vector.

The term “vector”, “expression vector”, or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as a gene encoding a protein of interest, one or more protein tags, functional domains and the like.

Vectors suitable for use in preparation of proteins and/or antigenic protein include those selected from baculovirus, phage, plasmid, phagemid, cosmid, fosmid, bacterial artificial chromosome, viral DNA, Pl-based artificial chromosome, yeast plasmid, and yeast artificial chromosome. For example, the viral DNA vector can be selected from vaccinia, adenovirus, foul pox virus, pseudorabies and a derivative of SV40. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.”

Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

In a further embodiment, the present invention provides an isolated cell with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence. In one aspect, the cell is a mammalian cell. In certain aspects, the mammalian cell is a human cell.

In one embodiment, the present invention provides a delivery vehicle with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence. In one aspect, the nucleic acid delivery vehicle is an extracellular vesicle (EV), a lipid nanoparticle (LNP), a liposome, a plasmid, or a viral vector.

As used herein, the terms “extracellular vesicle” and “ECV” or “EV” are interchangeable and refer to secreted cell-derived vesicles surrounded by a distinct phospholipid bilayer.

Exosomes are a family of small nanoparticles having a diameter comprised between 30 and 100 nm. Exosomes are generated inside multivesicular endosomes or multivesicular bodies (MVBs) and are secreted when these compartments fuse with plasma membrane. Exosomes are enriched in endosome-derived components and also contain many bioactive molecules such as proteins, lipids, and nucleic acids including mRNAs, microRNAs (miRNAs), long noncoding RNAs (lncRNAs), transfer RNA (tRNA), genomic DNA, cDNA, and mitochondrial DNA (mtDNA). Exosomes may be released from multiple cell types, including reticulocytes, immunocytes, infected cells, and cancer cells. Exosome secretion is a general cellular function that plays an important role in the intercellular transfer of information, as such exosomes exert their function through paracrine effects. These secreted vesicles serve as cell-to-cell messengers that modify cellular function in normal state as well as in disease state. Exosome resist degradation and contain membrane proteins potentially useful for targeting and docking. The most intriguing feature of exosomes is that they can be isolated from cultured cells and be subcutaneously administered, making exosomes an exciting therapeutic delivery system. Indeed, exosome products are in Phase I and II clinical trials for a variety of indications. Originally considered only a waste disposal system, they are now emerging as another class of signal mediators. Exosomes are secreted by any cell type and retrieved in every body fluid, such as blood, urine, saliva and amniotic liquid.

In an aspect, the EV is an exosome or a microvesicle.

In certain aspects, the viral vector is an adenoviral vector, an adeno-associated vector (AAV), or a lentiviral vector.

Polynucleotides can be delivered to cells (e.g., a plurality of different cells or cell types including target cells or cell types and/or non-target cell types) in a vector (e.g., an expression vector). Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viral vectors have several advantages for delivery of nucleic acids, including high infectivity and/or tropism for certain target cells or tissues. In some cases, a viral vector can be used to deliver a polynucleotide described herein.

In some aspects, the vector is an AAV vector. The term “AAV” is an abbreviation for adeno-associated virus and can be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, AAVDJ, rhlO, derivatives and hybrids thereof, avian AAV, bovine AAV, canine AAV, equine AAV, primate AAV, non-primate AAV, and ovine AAV. Additionally, any engineered or variant derived from ancestral AAV sequence reconstruction can be used as a vector. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

Techniques contemplated herein include polynucleotides delivery via a viral vector (e.g., retroviral, adenoviral, AAV, helper-dependent adenoviral systems, hybrid adenoviral systems, herpes simplex, pox virus, lentivirus, and Epstein-Barr virus), and non-viral systems, such as physical systems (naked DNA, DNA bombardment, electroporation, hydrodynamic, ultrasound, and magnetofection), and chemical systems (cationic lipids, different cationic polymers, and lipid polymers).

In a specific aspect, the delivery vehicle is an EV. In an additional aspect, the engineered antigenic protein is configured to elicit a humoral immune response and/or a cellular immune response in an animal subject.

The term “immune response” refers to an integrated bodily response to an antigen and preferably refers to a cellular immune response or a cellular as well as a humoral immune response. The immune response may be protective/preventive/prophylactic and/or therapeutic.

The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. B cells or B lymphocytes are a type of lymphocyte in the humoral immunity of the adaptive immune system and are important for immune surveillance. T cells or T lymphocytes are a type of lymphocyte that plays a central role in cell-mediated immunity. There are two major subtypes of T cells: the killer T cell and the helper T cell. In addition, there are suppressor T cells which have a role in modulating immune response. Killer T cells only recognize antigens coupled to Class I MHC molecules, while helper T cells only recognize antigens coupled to Class II MHC molecules. These two mechanisms of antigen presentation reflect the different roles of the two types of T cell. A third minor subtype are the γδ T cells that recognize intact antigens that are not bound to MHC receptors. In contrast, the B cell antigen-specific receptor is an antibody molecule on the B cell surface and recognizes whole pathogens without any need for antigen processing. Each lineage of B cell expresses a different antibody, so the complete set of B cell antigen receptors represent all the antibodies that the body can manufacture.

A “cellular immune response”, a “cellular response”, a “cellular response against an antigen” or a similar term is meant to include a cellular response directed to cells characterized by presentation of an antigen with class I or class II MHC. The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as infected cells, preventing the production of more diseased cells.

The terms “immunoreactive cell” “immune cells” or “immune effector cells” in the context of the present invention relate to a cell which exerts effector functions during an immune reaction. An “immunoreactive cell” preferably is capable of binding an antigen or a cell characterized by presentation of an antigen, or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

In certain aspects, the animal subject is a human subject.

In an additional embodiment, the present invention provides a pharmaceutical composition having a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence and a physiologically acceptable excipient and/or adjuvant.

By “pharmaceutically acceptable” it is meant the carrier, diluent, excipient or adjuvant must be compatible with the other ingredients of the formulation and not deleterious to the recipient thereof. Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

As used herein, an “adjuvant” refers to a substance that increases or modulates the immune response to an immunogenic composition, such as a vaccine for example. An immunologic adjuvant is defined as any substance that acts to accelerate, prolong, or enhance antigen-specific immune responses when used in combination with specific vaccine antigens. Adjuvants in immunology are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called pathogen-associated molecular patterns, which include liposomes, lipopolysaccharide, molecular cages for antigens, components of bacterial cell walls, and endocytosed nucleic acids such as RNA, double-stranded RNA, single-stranded DNA, and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells, lymphocytes, and macrophages by mimicking a natural infection.

There are many types of adjuvants, including inorganic compounds (e.g., potassium alum, aluminum hydroxide, aluminum phosphate, calcium phosphate hydroxide), oils: (e.g., paraffin oil, propolis, Adjuvant 65 (based on peanut oil)), bacterial products (e.g., killed bacteria of the species Bordetella pertussis, Mycobacterium bovis, toxoids), plant saponins from Quillaja, soybean, Polygala senega, cytokines (e.g., IL-1, IL-2, IL-12), combinations: (e.g., Freund's complete adjuvant, Freund's incomplete adjuvant), and other organic substances (e.g., squalene).

Alum was the first aluminium salt used as an adjuvant but has been almost completely replaced by aluminium hydroxide and aluminium phosphate for commercial vaccines. Aluminium salts are the most commonly used adjuvants in human vaccines. Alum can trigger dendritic cells and other immune cells to secrete Interleukin 1 beta (IL-1β), an immune signal that promotes antibody production. Alum adheres to the cell's plasma membrane and rearranges certain lipids there. Spurred into action, the dendritic cells pick up the antigen and speed to lymph nodes, where they stick tightly to a helper T cell and presumably induce an immune response. A second mechanism depends on alum may be related to the killing of immune cells at the injection site.

Squalene is a naturally occurring organic compound used in human and animal vaccines. Squalene is an oil, made up of carbon and hydrogen atoms, produced by plants and is present in many foods. Squalene is also produced by the human liver as a precursor to cholesterol and is present in human sebum. MF59 is an oil-in-water emulsion of squalene adjuvant used in some human vaccines. Over 22 million doses of a vaccine with squalene have been administered with no safety concerns. ASO3 is another squalene-containing adjuvant.

The plant extract QS-21 is a liposome made up of plant saponins from Quillaja saponaria, the soap bark tree.

Monophosphoryl lipid A (MPL), a detoxified version of Salmonella minnesota lipopolysaccharide, interacts with the receptor TLR4 to enhance immune response.

In a further aspect, the present invention provides a pharmaceutical composition having a delivery vehicle with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence and a physiologically acceptable excipient and/or adjuvant. In one aspect, the delivery vehicle has an EV and a chemical lipofection reagent or a chemical transfection reagent. In a specific aspect, the EV is an exosome or a microvesicle. In an additional aspect, the chemical lipofection reagent or the chemical transfection reagent is a polycationic lipid. In a further aspect, the polycationic lipid is an mRNA lipofection reagent or an mRNA transfection reagent.

In one embodiment, the present invention provides a method of making a nucleic acid delivery vehicle by loading a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence into an extracellular vesicle (EV). In one aspect, the EV is an exosome or a microvesicle. In an additional aspect, the nucleic acid molecules are pre-mixed with a chemical lipofection reagent or a chemical transfection reagent. In certain aspects, the chemical lipofection reagent or the chemical transfection reagent is a polycationic lipid. In some aspects, the polycationic lipid is an mRNA lipofection reagent or an mRNA transfection reagent.

In an additional embodiment, the present invention provides a method comprising administering into an animal a pharmaceutical composition having a delivery vehicle with a nucleic acid sequence encoding a protein having an extracellular domain, a transmembrane domain and a carboxy-terminal cytoplasmic domain, wherein the carboxy-terminal cytoplasmic domain have a deletion or addition of a heterologous sequence for improved cell-surface expression of the protein relative to the protein prior to the deletion or addition of the sequence and a physiologically acceptable excipient and/or adjuvant.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In one aspect, the administration elicits an antigenic immune response in a subject. In an additional aspect, the animal is a mammal. In a specific aspect, the mammal is a human. In a further aspect, the antigen specific immune response comprises a T cell response and/or a B cell response. In one aspect, the administration is a single administration. In an additional aspect, there is a booster dose.

In one embodiment, the present invention provides methods to increase the expression of membrane proteins in the surface of cells, exosomes and other extracellular vesicles. In one aspect, the method involves the simultaneous engineering of two general changes to the protein of interest, removal of retrieval signals and addition of ER export signals. More specifically, this invention involves the following changes to the protein of interest. The protein is modified so as to remove retrieval and targeting signals that serve to localize the protein of interest (or proteins generally) to specific compartments of the cell (e.g. the endoplasmic reticulum (ER), Golgi, endosome, lysosome, etc.), either by amino acid substitution mutations or the deletion of the protein's cytoplasmic regions (C-terminal tail in the case of type-1 membrane proteins) and the protein is also modified by the addition of an ER export signal, which may be accomplished by appending one or more ER export signals to one or more cytoplasmic regions of the protein, such as to the C-terminus of a type-I membrane protein; these peptides drive interaction with the COPII coat complex. Examples include VSV-G [-KLKHTKKRQIYTDIEMNRLGK_{COOH}] (SEQ ID NO:3), which we improved upon by substitution of alanine for a tyrosine within this peptide [-KLKHTKKRQIATDIEMNRLGK_{COOH}] (SEQ ID NO:6), a Y-to-A substitution that eliminates the clathrin-interacting YxxΦ motif in this peptide or expressing the extracellular domain(s) of the protein of interest as a fusion protein to a C-terminally positioned glycosylphosphatidylinositol (GPI) anchor-conferring peptide, such as the GPI anchor from CD55 [-PNKGSGTTSGTTRLLSGHTCFTLTGLLGTLVTMGLLT_{COOH}] (SEQ ID NO:7). This approach takes advantage of the fact that GPI anchors act as ER export signals through their binding to p24 protein family members.

In an additional embodiment, it is shown that combining the invention described herein (removal of retention motifs and appending of optimized ER export signals) with a third surface-directing change (i.e., the diproline substitution mutation) drives even higher cell surface expression of the protein of interest, a point established through studies of cell surface expression of SARS-CoV-2 Spike proteins.

In one embodiment, it is shown that use of the ER export signal-containing peptides [-KLKHTKKRQIATDIEMNRLGK_{COOH}] (SEQ ID NO:6) or [-KLKHTKKRQIYTDIEMNRLGK_{COOH}] (SEQ ID NO:3) leads to a net increase in the expression of the proteins to which they are appended.

In an additional embodiment, the method also enhances the expression of membrane proteins on the surface of exosomes and other extracellular vesicles, for any and all proteins and purposes, and for exosomes/extracellular vesicles derived from all cell lines.

In one embodiment, the method also reduces or eliminates the cytopathic effects of Spike expression, as these deleterious effects of Spike expression are linked to its sorting to lysosomes and are impaired by redirecting Spike to the cell surface. Method to specifically improve the cell surface expression of SARS-CoV-2 Spike.

### EMBODIMENTS

1. This invention can be used to increase the expression of membrane proteins on the surface of: cells AND exosomes and other extracellular vesicles.

2. This method involves the simultaneous engineering of two general changes to the protein of interest, removal of retrieval signals and addition of ER export signals. More specifically, this invention involves the following changes to the protein of interest:
a. the protein is modified so as to remove retrieval and targeting signals that serve to localize the protein of interest (or proteins generally) to specific compartments of the cell (e.g. the endoplasmic reticulum (ER), Golgi, endosome, lysosome, etc.), either by amino acid substitution mutations; OR deletion of the protein's cytoplasmic regions (C-terminal tail in the case of type-1 membrane proteins);
AND
b. the protein is also modified by the addition of an ER export signal, which may be accomplished by:
i. appending one or more ER export signals to one or more cytoplasmic regions of the protein, such as to the C-terminus of a type-I membrane protein; these peptides drive interaction with the COPII coat complex. Examples include VSV-G [-KLKHTKKRQIYTDIEMNRLGK_{COOH}] (SEQ ID NO:3), which was improved upon by substitution of alanine for a tyrosine within this peptide [-KLKHTKKRQIATDIEMNRLGK_{COOH}] (SEQ ID NO:6), a Y-to-A substitution that eliminates the clathrin-interacting YxxΦ motif in this peptide;
OR
ii. expressing the extracellular domain(s) of the protein of interest as a fusion protein to a C-terminally positioned glycosylphosphatidylinositol (GPI) anchor-conferring peptide, such as the GPI anchor from CD55 [-PNKGSGTTSGTTRLLSGHTCFTLTGLLGTLVTMGLLT_{COOH}] (SEQ ID NO:7). This approach takes advantage of the fact that GPI anchors act as ER export signals through their binding to p24 protein family members.

In the course of developing this invention, it was also showed for the first time that combining the invention described herein (removal of retention motifs and appending of optimized ER export signals) with a third surface-directing change (i.e., the diproline substitution mutation) drives even higher cell surface expression of the protein of interest, a point established by us through the studies of cell surface expression of SARS-CoV-2 Spike proteins

Furthermore, it was show that use of the ER export signal-containing peptides [-KLKHTKKRQIATDIEMNRLGK_{COOH}] (SEQ ID NO:6) or [-KLKHTKKRQIYTDIEMNRLGK_{COOH}] (SEQ ID NO:3) leads to a net increase in the expression of the proteins to which they are appended.

This method also enhances the expression of membrane proteins on the surface of exosomes and other extracellular vesicles, for any and all proteins and purposes, and for exosomes/extracellular vesicles derived from all cell lines.

Use of this method to improve cell surface expression for the purpose of enhancing immunogenicity and efficacy of proteins encoded by any expression-dependent vaccine (i.e. a vaccine that delivers protein-encoding nucleic acids (RNA or DNA), including mRNA vaccines, DNA vaccines, virus-encoded vaccines, cell-encoded vaccines, etc.).

This method also reduces or eliminates the cytopathic effects of Spike expression, as these deleterious effects of Spike expression are linked to its sorting to lysosomes and are impaired by redirecting Spike to the cell surface. Method to specifically improve the cell surface expression of SARS-CoV-2 Spike.

The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Differential Trafficking of Antigenetically Distinct Forms of Spike

Spike proteins are known to oscillate between multiple conformations. Given that protein sorting pathways and human antibody responses can both distinguish between different conformations of a given protein, it was tested whether conformational variants of Spike might be localized to different compartments of the human cell, and potentially identifiable by a subset of COVID-19 patient plasmas. Specifically, immunofluorescence microscopy was performed on doxycycline-induced Htet1/SW1 112 cells, which carry a doxycycline-inducible Spike gene encoding SW1, which has the same predicted amino acid sequence as Spike encoded by the SARS-CoV-2 reference strain Wuhan-Hu-1. Cells were fixed, permeabilized, and stained with a monoclonal antibody specific for (i) GM130 to visualize the Golgi and (ii) plasmas from Spike-reactive COVID-19 patients to visualize the subcellular distribution of Spike (green). These cells traffic SW1 to the plasma membrane as well as to the Golgi, shown here by the co-localization of Spike and GM130 in a subset of patient plasmas. However, it was found that using certain COVID-19 patient plasmas, especially plasmas 5 and G4, a 7 subpopulation of Spike protein was detected in large, non-Golgi structures of unknown identity.

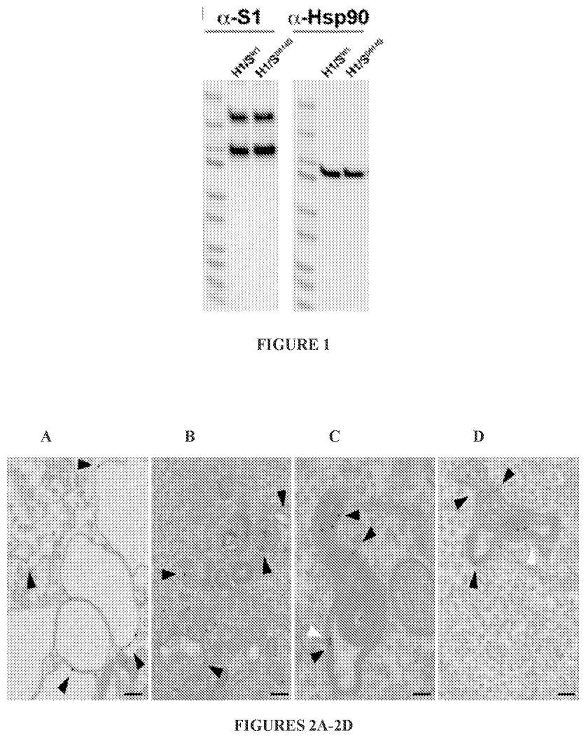

The potential significance of this observation was amplified further when these experiments were repeated in doxycycline-induced Htet1/SD614G cells, which express the D614G form of Spike (glycine at position 614 instead of aspartate). In contrast to what was observed in SW1-expressing cells, all four COVID-19 patient plasmas detected the large Spike-containing structures in doxycycline-induced Htet1/SD614G 128 cells. Given that both cell lines express similar levels of Spike and process it to a similar extent (FIG. 1), it was concluded that the D614G mutation alters Spike protein conformation in ways that either (i) alter its recognition by anti-Spike antibodies present in COVID-19 patient plasmas and/or (ii) change its intracellular trafficking within the cell.

### Example 2

### Spike is Trafficked to Lysosomes

To determine the identity of this compartment doxycycline-induced Htet1/SD614G 136 cells were stained with antibodies specific for organelle marker proteins, counter-staining the cells with plasma G4 to detect Spike. Multiple lysosomal proteins were enriched in these structures, including LAMP1, LAMP2, LAMP3/CD63, and mTOR. Marker of other organelles were not enriched in these structures, including those of the ER (calnexin and BiP), ERGIC (ERGIC 53 and ERGIC3,) Golgi (GM130), endosomes (EEA1), or plasma membrane (CD81). Ultrastructural studies confirmed the presence of Spike (12 nm gold) in the limiting membrane of various lysosome-like structures, including clusters of large empty lysosomes (FIG. 2A), clusters of small, 8 electron-dense lysosomes (FIG. 2B), and multilamellar endo/lysosomal compartments (FIG. 2C, D). Spike localization to lysosome membranes was also apparent following treatment with the PIKfyve inhibitor vacuolin-1, which inhibits lysosome fission and induces lysosome swelling.

### Example 3

### D614G Enhances Spike Trafficking to Lysosomes

To determine whether the D614G mutation affects the trafficking of Spike to lysosomes, we generated an antibody that binds to its C-terminal cytoplasmic tail, specifically, its C terminal 14 amino acids (-DSEPVLKGVKLHYT_{COOH}) (SEQ ID NO:8), which lies on the opposite side of the membrane from the D614G mutation. This antibody (rabbit S.C14) was affinity purified, found to be Spike-specific (FIG. 3A), and used to compare the subcellular distributions of SW1 and SD614G. These experiments revealed that the D614G mutation enhanced the lysosomal accumulation of Spike by ˜50% (p=2×10-7) (FIG. 3B).

### Example 4

### D614G Drives Spike Tolysomes in Virus Infected Cells

To determine whether the D614G mutation impacts the intracellular sorting of Spike in the more physiologically normal context of a virus infection, we infected Vero E6/TMPRSS2 cells at a multiplicity of infection (MOI) of 10 infectious particles per cell with a WT strain of SARS-CoV-2 (HP76) or a D614G strain of SARS-CoV-2 (HP7). These cultures were then incubated overnight (18 hours at 37° C.) to ensure that all cells were infected and producing SARS-CoV-2 particles. The two cell populations were then fixed, permeabilized, and stained using affinity-purified S.C14 9 antibodies, a monoclonal antibody specific to Lamp2 and DAPI to stain the nucleus.

Cells infected with the WT HP76 virus exhibited a diffuse distribution of Spike, with notable staining for Spike at the plasma membrane and other structures located far from Lamp2-positive lysosomes, with relatively little co-localization with Lamp2 and few instances of Spike-positive particles within Lamp2-positive membranes. In contrast, cells infected with the D614G virus HP7 localized Spike to small, intracellular structures clustered in the perinuclear area of the cell, most of which were located proximal to Lamp2-containing membranes, co-localized with Lamp2, or were encircled by Lamp2-containing membranes.

### Example 5

### Spike Trafficking to Lysosomes is Mediated by its Extracellular Domain

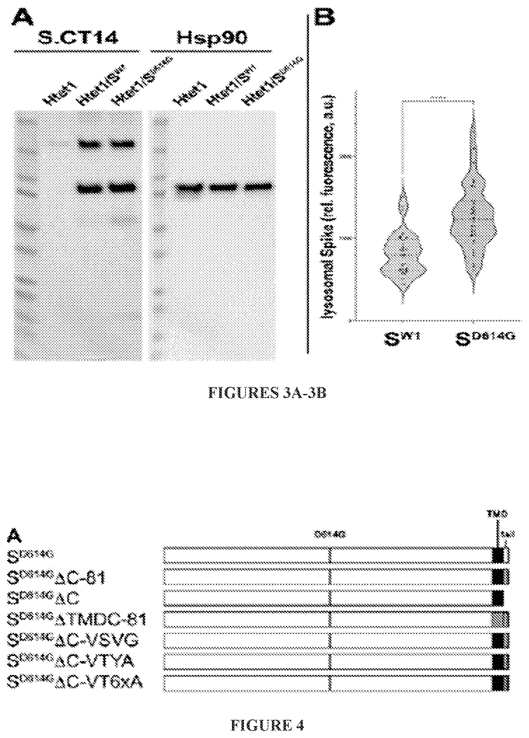

To better understand Spike trafficking, it was first tested whether it, like so many other lysosomal membrane proteins, carried its lysosomal sorting information in its C-terminal tail (aa 1256-1273; KFDEDDSEPVLKGVKLHYT_{COOH}) (SEQ I1) NO:5). This does not appear to be the case, as replacing its C-terminal 18 amino acids with the tail peptide of CD81 (-RNSSVYCOOH) did not block Spike trafficking to lysosomes (FIG. 4A), nor did deleting its C-terminal 18 amino acids. In fact, a Spike protein (SD614G DTMDC-81T) in which its entire transmembrane domain and tail (amino acids 1212-1273) were replaced with those from CD81 (-SGKLYLIGIAAIVVAVIMIFEMILSMVLCCGIRNSSVY_{COOH}) (SEQ ID NO:9) was also sorted to lysosomes. Is has been shown that an ER export signal in the C-terminal tail of SARS Spike and its presence in SARS-CoV-2 Spike was recently confirmed (Jennings et al., 2021). To explore the possibility that Spike residency in the ER contributes to its lysosomal localization, the C-terminal 18 amino acids of Spike was replaced with the 29 amino acid-long tail of VSV-G (-KLKHTKKRQIYTDIEMNRLGK_{COOH}) (SEQ ID NO:3), which contains a diacidic COPII-binding motif and functions as an ER export signal. The resulting protein, SD614G 197 DC-VT, showed far less co-localization with Lamp2 and greater localization at the plasma membrane, and similar results were observed for SD614G DC-VTYA (KLKHTKKRQIATDIEMNRLGK_{COOH}) (SEQ ID NO:6), which substitutes an alanine for the essential tyrosine (Ohno et al., 1995) of the endocytosis signal motif (YxxF) present within this peptide. A hexa-alanine substituted form of the VSVG tail peptide (KLKHTKKRQIAAAAAANRLGK_{COOH}) (SEQ ID NO:10) (Sevier et al., 2000) failed to override the lysosomal sorting of Spike.

To test whether Spike trafficking to lysosomes was sensitive to endocytosis inhibitors, we incubated Htet1/SD614G 206 cells in high concentrations of dynasore or pitstop2, added doxycycline to induce the expression of Spike, and examined the cells 20 hours later by immunofluorescence microscopy. Neither had a noticeable impact on the trafficking of Spike to lysosomes. Microtubules are also implicated in the trafficking of certain lysosomal cargoes but the microtubule depolymerizing agent nocodazole also had no effect. Lysosome biogenesis and function are more directly inhibited by bafilomycin, which blocks the proton-pumping vacuolar ATPase (V-ATPase) and prevents lysosome acidification. Bafilomycin redirected Spike into the Golgi and plasma membranes, preventing its accumulation in lysosomes.

### Example 6

### Spike and the D614G Mutation Disrupt Endomembrane Homeostasis

Coronavirus infection leads to dramatic changes in lysosome function, converting lysosomes from degradative organelles to viral storage depots and egress compartments. In this context, the evolution of Spike towards a lysosomal localization raises the possibility that Spike might be more than just a passive bystander in virus-producing cells. To explore this possibility, the effect of Spike expression on lysosome clustering was examined, a phenotype with diverse physiological effects. Lysosome clustering was infrequent in doxycycline-induced Htet1 cells, which exhibited a lysosome clustering score ([# of Lamp2-positive structures>2 μm in diameter]/[# of nuclei], collected across >10 randomly selected images) of 4% (40/970). In contrast, doxycycline-induced Htet1/SW1 227 cells exhibited a lysosome clustering score of 14% (159/1187) and doxycycline-induced Htet1/SD614G cells had a lysosome clustering score of 43% (507/1192). These results demonstrate that Spike expression alone is sufficient to induce lysosome clustering and that the D614G mutation amplified this phenotype by ˜4-fold ([43-4]/[14-4]).

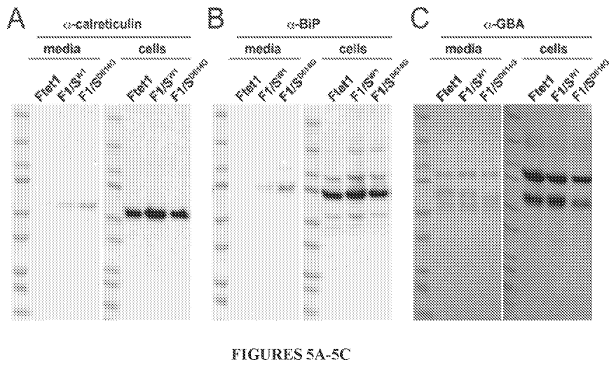

It was next tested whether Spike might also be sufficient to induce the coronavirus-induced secretion of KDEL-containing, resident ER enzymes. Cell lysates and soluble secreted proteins were collected from cultures of doxycycline-induced Ftet1, Ftet1/SW1, and Ftet1/SD614G cells and processed for immunoblot using antibodies specific for the ER-localized, KDEL-containing proteins calreticulin and BiP. Spike expression induced the secretion of both, and moreover, the D614G mutation amplified this effect by ˜4-fold (FIG. 5A, B). Spike expression had no impact on the secretion of B glucocerebrosidase, a lysosomal enzyme (FIG. 5C), demonstrating that Spike expression does not lead to a generalized increase in endomembrane exocytosis.

To test whether Spike expression also induced the lysosomal accumulation of KDEL receptors, doxycycline-induced Htet1/SD614G cells were stained using antibodies specific for the KDEL receptor and plasma G4, revealing that Spike expression is also sufficient to mediate this phenotype of coronavirus-infected cells. Finally, it was observed that the presence of Spike in lysosome membranes may impairs their uptake of endocytosed materials, as the labeling of doxycycline-induced Htet1/SD614G cells with AlexaFluor-647-labeled dextran (A647dextran) failed to lead to its uptake into Spike1 containing endolysosomal compartments.

### Example 7

### D614G Drives a 3-Fold Reduction in Cell Surface Spike Expression

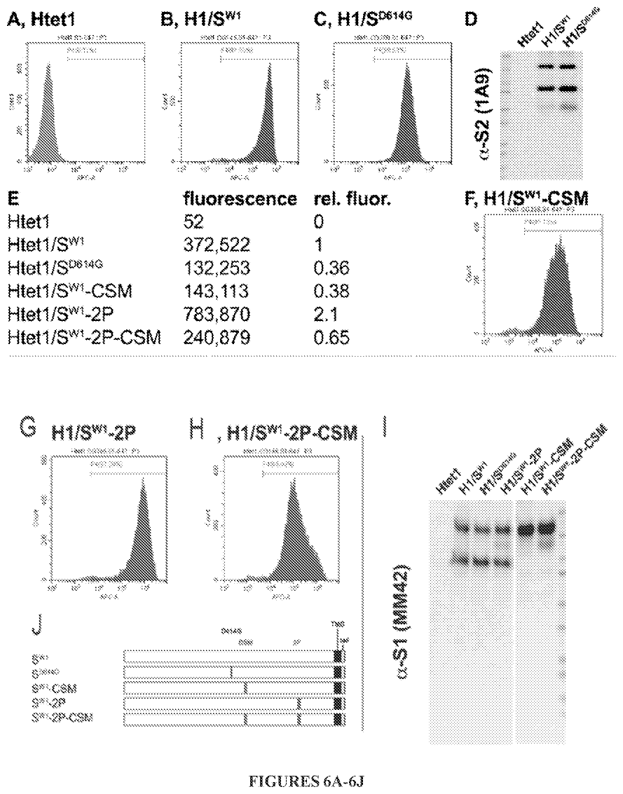

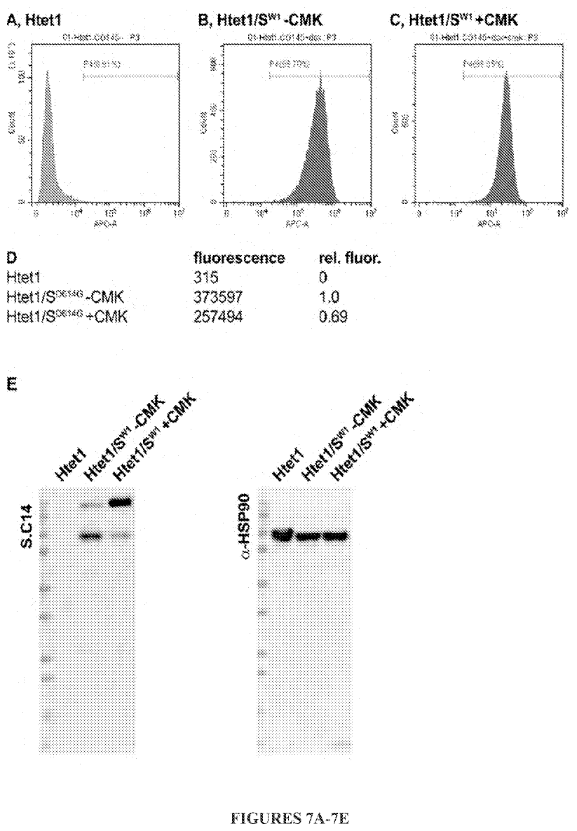

Protein trafficking is a zero-sum game, and as a result, the D614G-mediated increase in Spike accumulation in lysosomes must coincide with a drop in Spike expression at its other terminal destination, the plasma membrane. To determine the magnitude of this effect, flow cytometry was performed on doxycycline-induced Htet1/SW1 and Htet1/SD614G cells (FIGS. 6A-C and E). These experiments revealed that the D614G mutation drove an approximately 3-fold reduction in cell surface Spike expression. The 1A9 antibody used in these experiments detected both forms of Spike equally (FIG. 6D).

### Example 8

### D614G Suppresses Trafficking Defects Caused by the FCS Insertion Mutation

Prior to the SARS-CoV-2 pandemic, an ancestor of SARS-CoV-2 acquired the 12 nt-long insertion mutation that inserted a furin cleavage site (FCS) near its S1/S2 processing site. To determine whether this FCS insertion mutation impacted the lysosomal sorting information in Spike's extracellular domain, the cell surface expression and intracellular sorting of SW1-CSM was examined, a form of Spike that maintains the same amino acid spacing of SW1 268 but cannot be cleaved by furin due to substitution of key arginine residues (682RRAR685-to-682GSAG685) (Huang et al., 2021). Flow cytometry of doxycycline induced Htet1/SW1-CSM cells demonstrated that inactivation of the furin cleavage site caused an ˜3-fold reduction in Spike's cell surface expression (FIG. 6E, F). This result appears to be the result of enhanced lysosomal sorting of SW1-CSM, as it displayed stronger co-localization with Lamp2 than SW1, and more closely resembled the lysosomal sorting of SD614G.

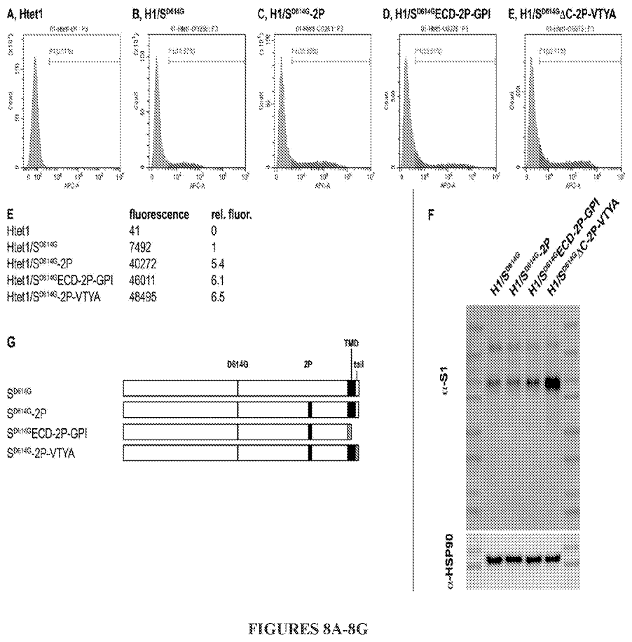

These results support a hypothesis in which acquisition of the FCS insertion mutation impaired the lysosomal trafficking of Spike, which was subsequently restored by the D614G mutation. Consistent with this hypothesis, addition of the furin inhibitor Decanoyl-RVKR-CMK to doxycycline-induced Htet1/SW1 cells reduced Spike's cell surface expression and furin-mediated processing (FIG. 7).

### Example 9

### The Diproline Mutation Increases Cell Surface Expression

The artificial diproline substitution (986KV987-to-986PP987) is the most biomedically important non-natural Spike mutation and was engineered into many expression dependent vaccines due to its stabilization of Spike in a trimeric, prefusion conformation and its association with higher induction of neutralizing antibody responses. However, the fact that this unselected substitution causes a pronounced change in Spike structure raises the possibility that it too may impair Spike's trafficking and lead to elevated cell surface Spike expression. This prediction was confirmed by flow cytometry of doxycycline-induced Htet1/SW1 291-2P cells, which demonstrated that the diproline substitution caused a 2-fold increase in cell surface Spike expression (FIG. 6E, F). This effect of was at least partly dependent on the presence of the furin cleavage site, as elimination of the furin cleavage site caused a 4-fold reduction in cell surface Spike expression (FIG. 6E, G). Immunoblot experiments confirmed that the CSM mutation had the expected effect on S1/S2 site processing (FIG. 6H).

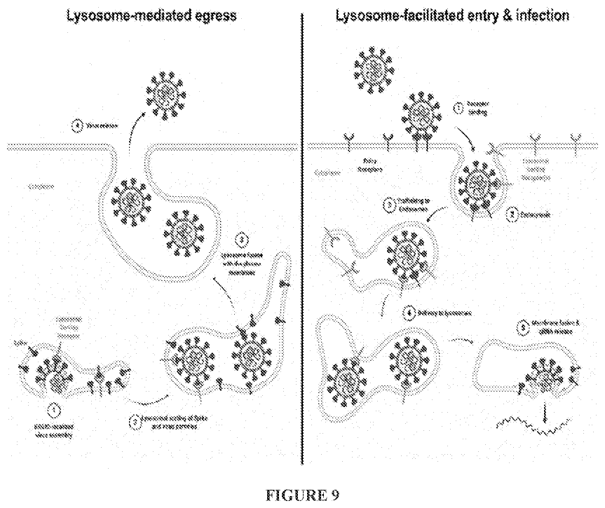

The FCS insertion mutation and the subsequent D614G mutation have become fixed in the SARS-CoV-2 genome, and as a result, all future vaccines will likely be composed of or express Spike proteins containing both of these mutations. Given that the immunogenicity of expression-dependent vaccines is positively correlated with the level 15 of cell surface antigen expression, it was tested whether the cell surface expression of S 304 D614G might be maximized by combining the diproline (2P) substitution with deletion of Spike's ER retrieval signal and addition of an ER export signal. Flow cytometry of transiently transfected Htet1 cells provides support for this hypothesis. Specifically, we observed that the diproline substitution was sufficient to induce a 5-fold increase cell surface expression of SD614G 308, and that combining the diproline substitution with deletion of its C-terminal tail and addition of ER export signals led to an even higher level of cell surface Spike expression (FIG. 8).

### Example 10

### Further Data

Flow cytometry of cells expressing different forms of the D614G form of SARS-CoV-2 Spike. The cell surface expression of Spike-D614G proteins was measured by flow cytometry of transfected HEK293 cells using an anti-Spike S2 monoclonal antibody (1A9).

TABLE 1

| | cell | | | integrated | relative cell |
|---|---|---|---|---|---|
| Spike protein | population | % expressing | mean fl | signal | surface expression |
| S/D614G | Htet1/CG258 | 20.06% | 33062.1 | 6632 | 1.0 |
| S/D614G-VT | Htet1/CG367 | 30.54% | 42523.1 | 12987 | 2.0 |
| S/D614G-VTYA | Htet1/CG370 | 31.85% | 50330.4 | 16030 | 2.4 |
| S/D614G(ECD)-GPI | Htet1/CG358 | 37.24% | 70522.1 | 26262 | 4.0 |
| S/D614G-VT6xA | Htet1/CG371 | 26.50% | 24179.5 | 6408 | 1.0 |

These data demonstrate that the invention described herein increased the cell surface expression of D614G Spike by 2.0-fold, 2.4-fold, and 4.0-fold, depending on whether the ER export signal employed in the study was the original VSV-G tail peptide (VT [-KLKHTKKRQIYTDIEMNRLGK_{COOH}]) (SEQ ID NO:3), the tyrosine-to-alanine substituted form of this peptide (VTYA, [-KLKHTKKRQIATDIEMNRLGK_{COOH}]) (SEQ NO:6), or the GPI anchor

Flow cytometry of cells expressing different forms of WT SARS-CoV-2 Spike. The cell surface expression of Spike-D614G proteins was measured by flow cytometry of transfected HEK293 cells using an anti-Spike S2 monoclonal antibody (1A9).

TABLE 2

| | cell | | | integrated | relative cell |
|---|---|---|---|---|---|
| Spike protein | population | % expressing | mean fl | signal | surface expression |
| S/WT | Htet1/CG256 | 26.35% | 73968.9 | 19491 | 1.0 |
| S/WT-2P | Htet1/CG262 | 40.54% | 193400.6 | 78405 | 4.0 |

These data demonstrate that the diproline substitution commonly incorporated into expression-dependent SARS-CoV-2 vaccines increases the cell surface expression of Spike by as much as 4-fold.

Flow cytometry of cells expressing different forms of WT SARS-CoV-2 Spike. The cell surface expression of Spike-D614G proteins was measured by flow cytometry of transfected HEK293 cells using an anti-Spike S2 monoclonal antibody (1A9).

TABLE 3

| | cell | | | integrated | relative cell |
|---|---|---|---|---|---|
| Spike protein | population | % expressing | mean fl | signal | surface expression |
| S/D614G | Htet1/CG258 | 18.67% | 40129.5 | 7492 | 1.0 |
| S/D614G-2P | Htet1/CG263 | 28.86% | 139543.5 | 40272 | 5.4 |
| S/D614G-2P(ECD)-GPI | Htet1/CG373 | 32.81% | 140235.2 | 46011 | 6.1 |
| S/D614G-2P-VTYA | Htet1/CG376 | 25.51% | 190102.1 | 48495 | 6.5 |

These data demonstrate that combining the changes specified by our invention (removal of ER retrieval signals and replacement with ER export signals) led to the highest cell surface expression of Spike D614G proteins, more than 6-fold greater than D614G alone.

### Example 11

### Methods

### Experimental Model and Subject Details

Viral Stocks. VeroE6/TMPRSS2 cells (Matsuyama et al., 2020) were used to grow and titrate infectious virus using established protocols (Klein et al., 2020; Schaecher et al., 2007). The clinical isolates SARS-CoV-2/USA/MD-HP00076/2020 (Spike D614; GenBank: MT509475.1) and SARS-Cov-2/USA/DC-HP00007/2020 (Spike G614; GenBank: MT509464.1) were isolated using published procedures (Gniazdowski et al., 2020) and virus stocks were grown on VeroE6/TMPRSS2 cells. Virus stocks were sequenced to confirm that the amino acid sequence of the isolate was identical to the sequence derived from the clinical sample.

Cell lines and culture conditions. VeroE6/TMPRSS2 cells, HEK293 cells and its Htet1 derivative, and 293F and its Ftet1 derivative, were maintained in complete medium (DMEM high glucose, with glutamine, containing 10% fetal bovine serum and 1% penicillin/streptomycin solution), supplemented with transgene-selecting antibiotics as needed, at 37° C., 90% H2O, and 5% C02. Caco-2 cells were cultured in DMEM high glucose, with glutamine, containing 20% fetal bovine serum and 1% penicillin/streptomycin solution. For measurement of Spike induced changes in KDEL protein secretion, Ftet1-derived cell lines were cultured in shaker flasks in chemically-defined media (Freestyle) at 110 rpm, 37° C., 90% H2O, and 8% CO2. Spike protein expression was in all cases induced by addition of doxycycline to the culture media at 1 μg/mL final concentration.

Human Plasmas. All patient plasmas were collected using standard procedures for blood draw and plasma collection. Following Johns Hopkins Medicine Institutional Review Board (IRB) approval, plasma samples were obtained under informed consent from healthy donors prior to the COVID-19 pandemic (JHM IRB NA_0004638)(Cox et al., 2005) and from COVID-19 patients, with specimens utilized for this publication obtained from the Johns Hopkins Biospecimen Repository, which is based on the contribution of many patients, research teams, and clinicians, and were collected following IRB approval (Johns Hopkins COVID 19 Clinical Characterization Protocol for Severe Infectious Diseases (IRB00245545) and Johns Hopkins COVID-19 Remnant Specimen Repository (IRB00248332)). All COVID 19 patient plasmas used in this study were collected on the day of admission of the patient into the Johns Hopkins Hospital, and between the dates of Apr. 7 and Apr. 22, 2020.

### Methods

Plasmid construction. The plasmid pS147 carries a single gene consisting of the CMV enhancer/promoter sequences upstream of a single ORF encoding (a) rtTAv16 (Das et al., 2016), (b) the porcine teschovirus 2a peptide, and (c) a codon-optimized bleomycin-resistance gene (Guo et al., 2021), followed by the expression-enhancing WPRE (Donello et al., 1998) and a polyadenylation signal. The plasmid pC is designed to drive expression of inserted transgenes from the CMV enhancer/promoter sequences on an mRNA carrying expression-enhancing WPRE (Donello et al., 1998) and a polyadenylation signal. Sleeping Beauty transposons were based on pITRSB (Guo et al., 2021) and carry (a) one transgene that drives expression of a puromycin-resistance gene (Guo et al., 2021) under the control of the EFS promoter, and (b) a second transgene containing the doxycycline inducible TRE3G promoter upstream of a Spike-expressing transgene (codon optimized) that encodes the Spike protein encoded by the SARS-CoV-2 reference strain (Zhou et al., 2020b)(NCBI Reference Sequence YP_009724390.1), which we refer to as SW1, or

derivatives of SW1 684 carrying the amino acid changes specified in the results section. All Spike genes were sequenced in their entirety and are identical to each other outside of specified coding differences.

DNA transfections and cell line creation. To make the doxycycline-inducible cell lines Htet1 and Ftet1, HEK293 and 293F cells (respectively) were transfected with the plasmid pS147, which expresses the Tet-on transcription factor rtTAv16 (Das et al., 2016). The next day the transfected cells were switched to zeocin-containing media, leading to the selection of hundreds of zeocin resistant cell clones, which were then pooled to generate polyclonal cell lines Htet1 and Ftet1. To make Spike-expressing derivatives of Htet1 or Ftet1, these cell lines were transfected, again using Lipofectamine according to the manufacturer's instructions, with Sleeping Beauty transposons designed to express a puromycin-resistance gene and a doxycycline-inducible Spike gene, the sequence of which was altered according to the needs of the experiment as designated in each section of the results. The next day, each transfected cell population was transferred to complete media containing both puromycin and zeocin. Ten days later, the surviving cell colonies were pooled, expanded, and used for the analysis of Spike protein trafficking and function. For transient transfections, Htet1 cells were transfected with the designated plasmids using Lipofectamine according to the manufacturer's instructions, while Caco-2 cells were transfected by electroporation according to the manufacturer's instructions (Neon, ThermoFisher).

Immunofluorescence microscopy. Cells were cultured on sterile, poly-L-lysine-coated cover glasses in complete media or in complete media containing drugs, vehicle, or probes. At the appropriate time point for each experiment, cells were fixed (4% formaldehyde in PBS) for 15 min, permeabilized (1% Triton X-100 in PBS) for 5 min and processed for immunofluorescence microscopy using established protocols. Cover glasses were mounted on slides using Fluoromount G. Cells were visualized using an EVOSM7000 fluorescence microscope equipped with 20× (PL FL 20×, 0.50NA/2.5WD), 40× (PLAN S-APO 40×, NA0.95, 0.18 MM), and 60× (OBJ PL APO 60×, 1.42NA/0.15WD) Olympus objectives. Confocal fluorescence microscopy was performed using a Zeiss LSM800 microscope with gallium-arsenide phosphide (GaAsP) detectors and a 100×/1.4na Plan-Apochromat objective. Images were assembled into figures using Adobe Photoshop and Adobe Illustrator. Quantitative analysis of image files was performed using ImageJ.

Electron Microscopy. Htet1/SD614G cells were grown in doxycycline-containing complete media on coated tissue culture plates for two days. The cells were then fixed with formaldehyde and glutaraldehyde, dehydrated, embedded in Epon, sectioned, interrogated with rabbit ant spike C-terminal peptide antibody and monoclonal anti-Lamp2 antibody, and washed. Sections were then incubated with 12 nm gold conjugates of goat anti-rabbit antibodies, and also with 12 nm gold conjugates of goat anti-rabbit antibodies and 6 nm gold conjugates of goat anti-mouse antibodies, washed, and stained with uranyl acetate. Sections were imaged on a Hitachi 7600 transmission electron microscope.

Immunoblot. Cells were grown in the presence or absence of doxycycline, lysed by addition of SDS PAGE sample buffer, boiled for 10 min, separated by SDS-PAGE, and transferred to PVDF membranes. Membranes were incubated with gentle rocking in blocking solution (5% nonfat dry milk in Tris-buffered saline, pH 7.4, containing 0.1% Tween-20 (TBST)) at room temperature for two hours, blocking solution containing primary antibodies overnight at 4° C., washed 5 times in TBST, incubated with horseradish peroxidase (HRP)-conjugated secondary antibodies in blocking solution for one hour at room temperature, then washed 5 times with TBST. Membranes were then incubated with Amersham ECL Western Blotting Detection Reagents to generate HRP-mediated chemiluminescence and visualized immediately using an Amersham Imager 600 gel imaging system.

Probe and drug treatments. Dextran labeling was performed by culturing coverglass-grown Htet1/SD614G 738 cells in complete media containing doxycycline and A647dextran (200 μg/mL final concentration), growing them for 20 hours, switching the cells back to complete media lacking A647dextran for three hours, then fixing the cells and processing the cells for immunofluorescence microscopy. To assess the effects of dynasore, pitstop2, nocodazole, and bafilomycin on spike protein trafficking, we switched coverglass-grown Htet1/SD614G cells from complete media to complete media containing doxycycline and each of these drugs at concentrations known to be inhibitory (50 μM for dynasore, 20 μM for pitstop2, 1 μM for nocodazole, and 43 nM for bafilomycin), grew them for 20 hours, and then fixed the cells and processed them for fluorescence microscopy. To examine spike expression in vacuolin-1-treated cells, coverglass-grown Htet1/SD614G cells were switched from complete media to complete media containing doxycycline, grown overnight to allow expression of spike, followed by addition of vacuolin-1 at a concentration of 10 μg/ml for three additional hours, and then fixed and processed for immunofluorescence microscopy. Protein secretion measurements Ftet1, Ftet1/SW1, and Ftet1/SD614G cells were seeded at a density of one million cells per mL in 150 mL shaker flasks and grown at a shaking speed of 110 rpm for a period of 3 days in 30 mL of FreeStyle™ 293 Expression Medium (ThermoFisher) supplemented with 1% penicillin/streptomycin solution and 1 μg/ml doxycycline. Cell pellets were collected by centrifugation at 300×g for 5 minutes and lysed in 2 mL of 2× SDS-PAGE sample buffer (with 2-mercaptoethanol, Halt protease inhibitor cocktail, and phosphatase inhibitor cocktails 2 & 3). For the conditioned media, large cell debris was removed by centrifugation at 3,000×g for 15 minutes, and the supernatant was passed through a 0.22 μm filter and spun at 100,000×g for 2 hours to remove extracellular vesicles. The resulting supernatant was concentrated to 100 ul by Centricon Plus-70 (10 kDa cutoff, Millipore, Cat #UFC701008) and lysed in 50 μL of 6× SDS-PAGE sample buffer (with 2-mercaptoethanol, Halt protease inhibitor cocktail, and phosphatase inhibitor cocktails 2 & 3) to generate the extracellular protein lysates for immunoblot analysis.

Flow Cytometry. Prior to the day of experiment, 100 μg of anti-SARS-CoV-2/S2 (1A9) antibody was conjugated to A647 using the Lightning-Link Conjugation Kit (Abcam, Cat #ab269823) according to the manufacturer's protocol. 0.5 million cells were seeded on a 6-well plate, grown in complete media containing doxycycline (1 μg/mL final concentration) for 20 hours, dissociated with TrypLE Express (ThermoFisher), passed through a cell-strainer 37cap (Falcon Cat #352235), transferred to an Eppendorf tube, and spun at 500 g for 4 min at 4° C. The resulting cell pellet was resuspended in 100 ul of chilled FACS buffer (1% FBS in PBS) containing 2 μL of the A647 conjugated 1A9 antibody and incubated on ice in dark for 30 min with gentle flicking every 10 min. Cells were then washed 3 times by adding 1 mL of chilled FACS buffer each time, spinning at 500 g for 4 min at 4° C., and discarding the supernatant. After the final wash, cells were resuspended in 250 μL of chilled FACS buffer containing 0.5 μg/mL of DAPI, incubated on ice in dark for 5 min, and analyzed using CytoFLEX S flow cytometer (Backman Coulter). Cells were gated based on (1) FSC-A vs SSC-A (P1), (2) FSC-A vs FCS-H (P2), (3) FSC-A vs PB450-A (P3), and (4) FSC-A vs APC-A (P4). Approximately 20,000 singlet, live cells (after gate P3) were recorded on the APC channel (A647 fluorescence), and the positive signals (after gate P4) were analyzed by % parent (P4/P3) and mean APC-A at P4.

### Quantification and Statistical Analysis

Image analysis. Images were analyzed using ImageJ, in combination with the Robust Automatic Threshold Selection (RATS) plugin. With RATS, we segmented both the Lamp2 and Spike immunostained channels. A binary mask was generated from each channel, and then colocalized regions were calculated as region of interest (ROI). The average gray value of ROI was measured.

Statistical analysis. Student's t test (two-tailed, unequal variances) was performed using GraphPad Prism version for Windows. p values are denoted as * where p<0.05; ** where p<0.01; where *** p<0.0002; **** where p<0.00001; and ns=not significant.

### Example 12

### Discussion

The data demonstrate that the earliest manifestation of the D614G mutation is to enhance the trafficking of Spike to lysosomes, the very organelle that serves as a storage depot for newly-synthesized coronavirus particles and mediates their egress from the cell. Here it was discussed how these findings advance our understanding of Spike protein distribution, its likely impact on immune responses to vaccines and infections, the roles of Spike in virus-producing cells, virus entry, and the evolutionary history of SARS-CoV-2.

### Spike Trafficking to Lysosomes

Most lysosomal membrane proteins contain lysosomal sorting signals in cytoplasmically-exposed regions of the protein. However, we demonstrated that (i) Spike's extracellular domain was sufficient to mediate its lysosomal localization, (ii) Spike's lysosomal localization was not blocked by replacement or deletion of Spike's cytoplasmic tail, and (iii) Spike trafficking was impacted by several mutations in Spike's extracellular domain. Not surprisingly, the Spike cytoplasmic tail lacks similarity to known lysosomal sorting signals (i.e., DXXLL, YXXØ, or [DE]XXX[LI]) and its lysosomal sorting was not blocked by small molecule inhibitors of the pathways by which they target membrane proteins to lysosomes. As for the mechanism that does mediate Spike trafficking to lysosomes, its elucidation rests upon the future identification of Spike's lysosomal sorting receptor(s).

### Spike Trafficking, D614G, and Immunity

It is a maxim that viruses evolve to minimize their cell-surface expression. In this context, the 3-fold reduction in Spike expression caused by the D614G mutation is likely to impact the immune responses to SARS-CoV-2 infection. Anti-Spike monoclonal antibody therapies are also likely to be affected by the relative cell surface expression of Spike, as their ability to induce antibody-dependent cytotoxicity and phagocytosis are dependent upon Spike expression by infected cells. However, the clearest implications of the study described herein are for expression-dependent vaccines, which rely on host cells to synthesize Spike and present it to the immune system. Antigenic presentation takes many forms, but there is clear evidence that the immunogenicity of expression-dependent vaccines is positively correlated with the extent of cell surface antigen expression. It was showed here that the diproline substitution (2P) causes in a 2-fold to 5-fold increase in cell surface Spike expression, and this effect may be a key factor in the superior immunogenicity of S-2P-expressing vaccines. Moreover, it was found that cell surface Spike expression could be maximized by combining the diproline substitution with deletion of the Spike C-terminus and the addition of an ER export signal. Whether this combination enhances the efficacy of expression-dependent vaccines is an open question, but one that should be explored in the development of next-generation Spike vaccines, as all variants of concern carry the D614G mutation.

### Spike and D614G Mediate Lysosome Dysfunction

The lysosomal accumulation of Spike does not lead to its enhanced degradation. Rather, it leads to Spike-mediated reprogramming of host endomembrane functions. Prior studies have established that coronavirus infection leads to numerous changes to lysosomal and ER homeostasis, including the conversion of lysosomes into virus release compartments. While some of these virus-induced changes are mediated by other viral proteins (i.e., the orf3a-mediated deacidification of lysosomes) but we show here that Spike is sufficient to drive several of these changes on its own, including lysosome clustering, the mislocalization of KDEL receptors to lysosomes, the aberrant secretion of KDEL enzymes, and perhaps even an impaired uptake of endocytosed materials. The D614G mutation appears to amplify these activities, indicating that they are affected by the lysosome-localized form of Spike. As for why coronaviruses exert such pleiotropic effects on host endomembrane systems, some appear to be facets of lysosome reprogramming required for viral egress. However, the Spike-induced mislocalization of KDEL receptors may have broader implications, as these G-protein coupled receptors link secretory cargo flux to the biogenesis of secretory organelles. While the mechanistic significance of these (and other) Spike-induced phenotypes remains to be established, our results clearly expand our understanding of Spike gene and protein function to include active roles in the modulation of infected, virus-assembling cells, in addition to its well-established roles in receptor binding and membrane fusion.

### D614G Suppresses Deleterious Traits of the FCS Insertion Mutation

Genetic suppressor screens are a powerful approach to understanding gene function. By extension, the identification of genetic suppressor relationships between known mutations of interest can yield equally impactful insights into gene function. The data presented in this report clearly establish that the D614G mutation is an intragenic suppressor of the 12 nt-long FCS insertion mutation. This genetic relationship is clear from the following observations:
(1) a Spike protein lacking the FCS, which models the Spike protein encoded by the SARS-CoV-2 ancestor, displayed strong lysosomal localization and low cell surface expression;
(2) insertion of the 12 nt-long FCS mutation impaired the lysosomal sorting of Spike and caused an ˜3-fold increase in its cell surface expression; and
(3) acquisition of the D614G mutation restored its trafficking to lysosomes and decreased its cell surface expression by ˜3-fold.

The reading of the available literature indicates that this is not the only FCS-associated phenotype suppressed by the D614G mutation, as a similar genetic relationship is evident from studies of TMPRSS2-independent infectivity:
(1) SARS-CoV-2 viruses that model its ancestor, and therefore lack the FCS insertion mutation, efficiently infect cells independent of TMPRSS2;
(2) acquisition of the FCS insertion mutation caused a dramatic drop in TMPRSS2-independent infectivity; and
(3) addition of the D614G mutation restores SARS-CoV-2's ability to infect cells by the TMPRSS2-independent pathway.

These observations represent strong evidence that the D614G is an intragenic suppressor of the FCS insertion mutation. Moreover, the fact that the D614G mutation is an intragenic suppressor of two separate phenotypes of the FCS insertion that manifest in two vastly different phases of the viral replication cycle, we propose here that these two processes share a common mechanistic basis. More specifically, it is proposed here that the D614G mutation reveals a shared mechanism between the lysosomal sorting of Spike and the TMPRSS2-independent pathway of virus infection, which is proposes to also involve the engagement of Spike with a lysosomal sorting pathway (Figure S6).

### A Lysosomal Model of Coronavirus Egress and Entry

The lysosomal model of SARS-CoV-2 egress and entry that emerges from the above considerations is easily reconciled with the data presented in this report, as well as with data from prior studies of coronavirus egress and entry. This is most obvious in the area of coronavirus biogenesis, which was already known to involve extensive reprogramming of lysosome function, leading to lysosomal accumulation of newly-synthesized virus particles and lysosome-mediated viral release. As for the hypothesis that the TMPRSS2-independent pathway of virus entry involves Spike-mediated engagement with a plasma membrane-to-lysosome transport pathway, this is admittedly more speculative but is at the same time consistent with many prior observations. These include the rapid appearance of virus particles in lysosomes shortly after the start of an infection, and the strong genetic and pharmacological evidence that coronavirus entry requires lysosomal proteases, the vacuolar ATPase, and endo/lysosomal transport factors. There is even some precedent for this model in the replication cycle of varicella zoster virus (VZV), which uses mannose-6-phosphate (M6P) and the M6P-dependent lysosomal protein sorting pathway to mediate VZV egress and VZV entry.

### Evolution of the D614G as an Intragenic Suppressor Mutation

This model of SARS-CoV-2 egress and entry allows one to see the evolution of the D614G mutation as a story of possession, loss, and restoration of Spike's lysosomal trafficking. In this narrative, the ancestor of SARS-CoV-2 expressed a Spike protein that was efficiently trafficked to lysosomes and facilitated efficient entry by a TMPRSS2-independent pathway of infection. However, the acquisition of the 12 nt-long FCS insertion mutation changed this dynamic by conferring numerous fitness-enhancing traits while at the same time hindering Spike trafficking to lysosomes. The result was a pleiotropic attenuation of Spike-mediated activities, an increase in cell surface Spike expression (and whatever immunological consequences this may cause), and impaired infection by the TMPRSS2-independent pathway of infection. These deleterious traits are rapidly selected against even in vitro, as FCS reversion mutations appear within just 2 rounds of SARS-CoV-2 replication in Vero cells. It is proposed here that these traits were also rapidly selected against in vivo, though in this case by their intragenic suppression via the D614G mutation rather than by reversion of the FCS insertion. In so doing, the D614G mutation allowed SARS-CoV-2 to retain the advantageous traits of the FCS insertion yet also restore the lysosomal trafficking of Spike the TMPRSS2-independent infectivity that had been lost as a consequence of the FCS insertion. As for the relative timing of the FCS insertion and D614G mutations, the data provided herein indicate that they likely occurred in rapid (replicative) succession, a notion supported by the absence of the FCS insertion in close evolutionary relatives of SARS-CoV-2 and the rise of the D614G mutation just weeks after the start of the COVID-19 pandemic. Furthermore, the simplicity of the D614G mutation (a 1 nt transition) and the superior transmission conferred by the FCS insertion in various animals and in human airway epithelial cells, are together indicative of FCS insertion occurring at or just prior to the zoonotic leap of SARS-CoV-2 into the human population.

### REFERENCES

Andersen, K. G., Rambaut, A., Lipkin, W. I., Holmes, E. C., and Garry, R. F. (2020). The proximal origin of SARS-CoV-2. Nat Med 26, 450-452. 10.1038/s41591-020-0820-9.
Baggen, J., Persoons, L., Vanstreels, E., Jansen, S., Van Looveren, D., Boeckx, B., Geudens, V., De Man, J., Jochmans, D., Wauters, J., et al. (2021). Genome-wide CRISPR screening identifies TMEM106B as a proviral host factor for SARS-CoV-2. Nat Genet 53, 435-444. 10.1038/s41588-021-00805-2.
Baravalle, G., Schober, D., Huber, M., Bayer, N., Murphy, R. F., and Fuchs, R. (2005). Transferrin recycling and dextran transport to lysosomes is differentially affected by bafilomycin, nocodazole, and low temperature. Cell Tissue Res 320, 99-113.10.1007/s00441-004-1060-x.
Barlowe, C. K., and Miller, E. A. (2013). Secretory protein biogenesis and traffic in the early secretory pathway. Genetics 193, 383-410. 10.1534/genetics.112.142810.
Bayer, N., Schober, D., Prchla, E., Murphy, R. F., Blaas, D., and Fuchs, R. (1998). Effect of bafilomycin A1 and nocodazole on endocytic transport in HeLa cells: implications for viral uncoating and infection. J Virol 72, 9645-9655. 10.1128/JVI.72.12.9645-9655.1998.
Braulke, T., and Bonifacino, J. S. (2009). Sorting of lysosomal proteins. Biochim Biophys Acta 1793, 605-614. 10.1016/j.bbamcr.2008.10.016.
Bu, Z., Ye, L., Vzorov, A., Taylor, D., Compans, R. W., and Yang, C. (2004). Enhancement of immunogenicity of an HIV Env DNA vaccine by mutation of the Tyr-based endocytosis motif in the cytoplasmic domain. Virology 328, 62-73. 10.1016/j.virol.2004.06.041.
Burkard, C., Verheije, M. H., Wicht, O., van Kasteren, S. I., van Kuppeveld, F. J., Haagmans, B. L., Pelkmans, L., Rottier, P. J., Bosch, B. J., and de Haan, C. A. (2014). Coronavirus cell entry occurs through the endo-/lysosomal pathway in a proteolysis-dependent manner. PLoS Pathog 10, e1004502. 10.1371/journal.ppat.1004502.
Cai, Y., Zhang, J., Xiao, T., Peng, H., Sterling, S. M., Walsh, R. M., Jr., Rawson, S., Rits-Volloch, S., and Chen, B. (2020). Distinct conformational states of SARS-CoV-2 spike protein. Science 369, 1586-1592. 10.1126/science.abd4251.
Cancino, J., Capalbo, A., Di Campli, A., Giannotta, M., Rizzo, R., Jung, J. E., Di Martino, R., Persico, M., Heinklein, P., Sallese, M., and Luini, A. (2014). Control systems of membrane transport at the interface between the endoplasmic reticulum and the Golgi. Dev Cell 30, 280-294. 10.1016/j.devcel.2014.06.018.
Cantuti-Castelvetri, L., Ojha, R., Pedro, L. D., Djannatian, M., Franz, J., Kuivanen, S., van der Meer, F., Kallio, K., Kaya, T., Anastasina, M., et al. (2020). Neuropilin-1 facilitates SARS-CoV-2 cell entry and infectivity. Science. 10.1126/science.abd2985.
Capitani, M., and Sallese, M. (2009). The KDEL receptor: new functions for an old protein. FEBS Lett 583, 3863-3871. 10.1016/j.febslet.2009.10.053.
Casadevall, A., Pirofski, L. A., and Joyner, M. J. (2021). The Principles of Antibody Therapy for Infectious Diseases with Relevance for COVID-19. mBio 12. 10.1128/mBio.03372-20.
Chen, J. J., Zhu, Z., Gershon, A. A., and Gershon, M. D. (2004). Mannose 6-phosphate receptor dependence of varicella zoster virus infection in vitro and in the epidermis during varicella and zoster. Cell 119, 915-926. 10.1016/j.cell.2004.11.007.
Corbett, K. S., Flynn, B., Foulds, K. E., Francica, J. R., Boyoglu-Barnum, S., Werner, A. P., Flach,
B., O'Connell, S., Bock, K. W., Minai, M., et al. (2020). Evaluation of the mRNA-Vaccine against SARS-CoV-2 in Nonhuman Primates. N Engl J Med 383, 1544-1555. 10.1056/NEJMoa2024671.
Corti, D., Purcell, L. A., Snell, G., and Veesler, D. (2021). Tackling COVID-19 with neutralizing monoclonal antibodies. Cell 184, 3086-3108. 10.1016/j.cell.2021.05.005.
Daly, J. L., Simonetti, B., Klein, K., Chen, K. E., Williamson, M. K., Anton-Plagaro, C., Shoemark, D. K., Simon-Gracia, L., Bauer, M., Hollandi, R., et al. (2020). Neuropilin-1 is a host factor for SARS-CoV-2 infection. Science. 10.1126/science.abd3072.
Daniloski, Z., Jordan, T. X., Wessels, H. H., Hoagland, D. A., Kasela, S., Legut, M., Maniatis, S., Mimitou, E. P., Lu, L., Geller, E., et al. (2020). Identification of Required Host Factors for SARS-CoV-2 Infection in Human Cells. Cell. 10.1016/j.cell.2020.10.030.
Das, A. T., Tenenbaum, L., and Berkhout, B. (2016). Tet-On Systems For Doxycycline-inducible Gene Expression. Curr Gene Ther 16, 156-167. 10.2174/1566523216666160524144041.
David-Ferreira, J. F., and Manaker, R. A. (1965). An Electron Microscope Study of the Development of a Mouse Hepatitis Virus in Tissue Culture Cells. J Cell Biol 24, 57-78. 10.1083/jcb.24.1.57.
Davidson, A. D., Williamson, M. K., Lewis, S., Shoemark, D., Carroll, M. W., Heesom, K. J., Zambon, M., Ellis, J., Lewis, P. A., Hiscox, J. A., and Matthews, D. A. (2020).
Characterisation of the transcriptome and proteome of SARS-CoV-2 reveals a cell passage induced in-frame deletion of the furin-like cleavage site from the spike glycoprotein. Genome Med 12, 68. 10.1186/si3073-020-00763-0.
Deigin, Y., and Segreto, R. (2021). SARS-CoV-2's claimed natural origin is undermined by issues with genome sequences of its relative strains: Coronavirus sequences RaTG13, MP789 and RmYN02 raise multiple questions to be critically addressed by the scientific community. Bioessays, e2100015. 10.1002/bies.202100015.
Donello, J. E., Loeb, J. E., and Hope, T. J. (1998). Woodchuck hepatitis virus contains a tripartite posttranscriptional regulatory element. J Virol 72, 5085-5092. 10.1128/JVI.72.6.5085-5092.1998.
Ducatelle, R., and Hoorens, J. (1984). Significance of lysosomes in the morphogenesis of coronaviruses. Arch Virol 79, 1-12. 10.1007/BF01314299.
Fregno, I., Fasana, E., Bergmann, T. J., Raimondi, A., Loi, M., Solda, T., Galli, C., D'Antuono, R., Morone, D., Danieli, A., et al. (2018). ER-to-lysosome-associated degradation of proteasome-resistant ATZ polymers occurs via receptor-mediated vesicular transport. EMBO J 37. 10.15252/embj.201899259.
Futai, M., Sun-Wada, G. H., Wada, Y., Matsumoto, N., and Nakanishi-Matsui, M. (2019). Vacuolar-type ATPase: A proton pump to lysosomal trafficking. Proc Jpn Acad Ser B Phys Biol Sci 95, 261-277. 10.2183/pjab.95.018.
Ghosh, S., Dellibovi-Ragheb, T. A., Kerviel, A., Pak, E., Qiu, Q., Fisher, M., Takvorian, P. M., Bleck, C., Hsu, V. W., Fehr, A. R., et al. (2020). beta-Coronaviruses Use Lysosomes for Egress Instead of the Biosynthetic Secretory Pathway. Cell. 10.1016/j.cell.2020.10.039.
Gniazdowski, V., Morris, C. P., Wohl, S., Mehoke, T., Ramakrishnan, S., Thielen, P., Powell, H., Smith, B., Armstrong, D. T., Herrera, M., et al. (2020). Repeat COVID-19 Molecular Testing: Correlation of SARS-CoV-2 Culture with Molecular Assays and Cycle Thresholds. Clin Infect Dis. 10.1093/cid/ciaa1616.
Guo, C., Fordjour, F. K., Tsai, S. J., Morrell, J. C., and Gould, S. J. (2021). Choice of selectable marker affects recombinant protein expression in cells and exosomes. J Biol Chem, 100838. 10.1016/j.jbc.2021.100838.
Hansen, G. H., Delmas, B., Besnardeau, L., Vogel, L. K., Laude, H., Sjostrom, H., and Noren, O. (1998). The coronavirus transmissible gastroenteritis virus causes infection after receptor-mediated endocytosis and acid-dependent fusion with an intracellular compartment. J Virol 72, 527-534. 10.1128/JVI.72.1.527-534.1998.
Hellerschmied, D., Serebrenik, Y. V., Shao, L., Burslem, G. M., and Crews, C. M. (2019). Protein folding state-dependent sorting at the Golgi apparatus. Mol Biol Cell 30, 2296-2308. 10.1091/mbc.E19-01-0069.
Hodgkin, J. (2005). Genetic suppression. WormBook, 1-13. 10.1895/wormbook.1.59.1. Hoffmann, M., Kleine-Weber, H., and Pohlmann, S. (2020a). A Multibasic Cleavage Site in the Spike Protein of SARS-CoV-2 Is Essential for Infection of Human Lung Cells. Mol Cell 78, 779-784 e775. 10.1016/j.molcel.2020.04.022.
Hoffmann, M., Kleine-Weber, H., Schroeder, S., Kruger, N., Herrler, T., Erichsen, S., Schiergens, T. S., Herrler, G., Wu, N. H., Nitsche, A., et al. (2020b). SARS-CoV-2 Cell Entry Depends on ACE2 and TMPRSS2 and Is Blocked by a Clinically Proven Protease Inhibitor. Cell 181, 271-280 e278. 10.1016/j.cell.2020.02.052.
Hogan, M. J., Conde-Motter, A., Jordan, A. P. O., Yang, L., Cleveland, B., Guo, W., Romano, J., Ni, H., Pardi, N., LaBranche, C. C., et al. (2018). Increased surface expression of HIV-1 envelope is associated with improved antibody response in vaccinia prime/protein boost immunization. Virology 514, 106-117. 10.1016/j.virol.2017.10.013.
Hou, Y., Meulia, T., Gao, X., Saif, L. J., and Wang, Q. (2019). Deletion of both the Tyrosine-Based Endocytosis Signal and the Endoplasmic Reticulum Retrieval Signal in the Cytoplasmic Tail of Spike Protein Attenuates Porcine Epidemic Diarrhea Virus in Pigs. J Virol 93. 10.1128/JVI.01758-18.
Hou, Y. J., Chiba, S., Halfmann, P., Ehre, C., Kuroda, M., Dinnon, K. H., 3rd, Leist, S. R., Schafer, A., Nakajima, N., Takahashi, K., et al. (2020). SARS-CoV-2 D614G variant exhibits efficient replication ex vivo and transmission in vivo. Science. 10.1126/science.abe8499.
Huang, K. A., Tan, T. K., Chen, T. H., Huang, C. G., Harvey, R., Hussain, S., Chen, C. P., Harding, A., Gilbert-Jaramillo, J., Liu, X., et al. (2021). Breadth and function of antibody response to acute SARS-CoV-2 infection in humans. PLoS Pathog 17, e1009352. 10.1371/journal.ppat.1009352.
Jennings, B. C., Kornfeld, S., and Doray, B. (2021). A weak COPI binding motif in the cytoplasmic tail of SARS-CoV-2 spike glycoprotein is necessary for its cleavage, glycosylation, and localization. FEBS Lett. 10.1002/1873-3468.14109.
Jia, R., and Bonifacino, J. S. (2019). Lysosome Positioning Influences mTORC2 and AKT Signaling. Mol Cell 75, 26-38 e23. 10.1016/j.molcel.2019.05.009.
Johnson, B. A., Xie, X., Bailey, A. L., Kalveram, B., Lokugamage, K. G., Muruato, A., Zou, J., Zhang, X., Juelich, T., Smith, J. K., et al. (2021). Loss of furin cleavage site attenuates SARS-CoV-2 pathogenesis. Nature 591, 293-299. 10.1038/s41586-021-03237-4.
Jungreis, I., Sealfon, R., and Kellis, M. (2021). SARS-CoV-2 gene content and COVID-19 mutation impact by comparing 44 Sarbecovirus genomes. Nat Commun 12, 2642. 10.1038/s41467-021-22905-7.
Kang, Y. L., Chou, Y. Y., Rothlauf, P. W., Liu, Z., Soh, T. K., Cureton, D., Case, J. B., Chen, R. E., Diamond, M. S., Whelan, S. P. J., and Kirchhausen, T. (2020). Inhibition of PIKfyve kinase prevents infection by Zaire ebolavirus and SARS-CoV-2. Proc Natl Acad Sci USA 117, 20803-20813. 10.1073/pnas.2007837117.
Ke, Z., Oton, J., Qu, K., Cortese, M., Zila, V., McKeane, L., Nakane, T., Zivanov, J., Neufeldt, C. J., Cerikan, B., et al. (2020). Structures and distributions of SARS-CoV-2 spike proteins on intact virions. Nature. 10.1038/s41586-020-2665-2.
Kirchdoerfer, R. N., Cottrell, C. A., Wang, N., Pallesen, J., Yassine, H. M., Turner, H. L., Corbett, K. S., Graham, B. S., McLellan, J. S., and Ward, A. B. (2016). Pre-fusion structure of a human coronavirus spike protein. Nature 531, 118-121. 10.1038/nature17200.
Klimstra, W. B., Tilston-Lunel, N. L., Nambulli, S., Boslett, J., McMillen, C. M., Gilliland, T., Dunn, M. D., Sun, C., Wheeler, S. E., Wells, A., et al. (2020). SARS-CoV-2 growth, furin-cleavage-site adaptation and neutralization using serum from acutely infected hospitalized COVID-19 patients. J Gen Virol 101, 1156-1169. 10.1099/jgv.0.001481.
Kokubun, H., Jin, H., and Aoe, T. (2019). Pathogenic Effects of Impaired Retrieval between the Endoplasmic Reticulum and Golgi Complex. Int J Mol Sci 20.10.3390/ijms20225614.
Korber, B., Fischer, W. M., Gnanakaran, S., Yoon, H., Theiler, J., Abfalterer, W., Hengartner, N., Giorgi, E. E., Bhattacharya, T., Foley, B., et al. (2020). Tracking Changes in SARS-CoV-2 Spike: Evidence that D614G Increases Infectivity of the COVID-19 Virus. Cell 182, 812-827 e819. 10.1016/j.cell.2020.06.043.
Krijnse-Locker, J., Ericsson, M., Rottier, P. J., and Griffiths, G. (1994). Characterization of the budding compartment of mouse hepatitis virus: evidence that transport from the RER to the Golgi complex requires only one vesicular transport step. J Cell Biol 124, 55-70. 10.1083/jcb.124.1.55.
Krzystyniak, K., and Dupuy, J. M. (1984). Entry of mouse hepatitis virus 3 into cells. J Gen Virol 65 (Pt 1), 227-231. 10.1099/0022-1317-65-1-227.
Lamers, M. M., Mykytyn, A. Z., Breugem, T. I., Wang, Y., Wu, D. C., Riesebosch, S., van den Doel, P. B., Schipper, D., Bestebroer, T., Wu, N. C., and Haagmans, B. L. (2021). Human airway cells prevent SARS-CoV-2 multibasic cleavage site cell culture adaptation. Elife 10. 10.7554/eLife.66815.
Lau, S. Y., Wang, P., Mok, B. W., Zhang, A. J., Chu, H., Lee, A. C., Deng, S., Chen, P., Chan, K. H., Song, W., et al. (2020). Attenuated SARS-CoV-2 variants with deletions at the S1/S2 junction. Emerg Microbes Infect 9, 837-842. 10.1080/22221751.2020.1756700.
Li, Y. C., Bai, W. Z., Hirano, N., Hayashida, T., and Hashikawa, T. (2012). Coronavirus infection of rat dorsal root ganglia: ultrastructural characterization of viral replication, transfer, and the early response of satellite cells. Virus Res 163, 628-635. 10.1016/j.virusres.2011.12.021.
Liu, Z., Zheng, H., Lin, H., Li, M., Yuan, R., Peng, J., Xiong, Q., Sun, J., Li, B., Wu, J., et al. (2020). Identification of Common Deletions in the Spike Protein of Severe Acute Respiratory Syndrome Coronavirus 2. J Virol 94. 10.1128/JVI.00790-20.
Lorenzo-Redondo, R., Nam, H. H., Roberts, S. C., Simons, L. M., Jennings, L. J., Qi, C., Achenbach, C. J., Hauser, A. R., Ison, M. G., Hultquist, J. F., and Ozer, E. A. (2020). A Unique Clade of SARS-CoV-2 Viruses is Associated with Lower Viral Loads in Patient Upper Airways. medRxiv. 10.1101/2020.05.19.20107144.
Marsh, M., and Pelchen-Matthews, A. (2000). Endocytosis in viral replication. Traffic 1, 525-532. 10.1034/j.1600-0854.2000.010701.x.
McBride, C. E., Li, J., and Machamer, C. E. (2007). The cytoplasmic tail of the severe acute respiratory syndrome coronavirus spike protein contains a novel endoplasmic reticulum retrieval signal that binds COPI and promotes interaction with membrane protein. J Virol 81, 2418-2428. 10.1128/JVI.02146-06.
Mellors, J., Tipton, T., Longet, S., and Carroll, M. (2020). Viral Evasion of the Complement System and Its Importance for Vaccines and Therapeutics. Front Immunol 11, 1450. 10.3389/fimmu.2020.01450.
Miller, E. A., Beilharz, T. H., Malkus, P. N., Lee, M. C., Hamamoto, S., Orci, L., and Schekman, R. (2003). Multiple cargo binding sites on the COPII subunit Sec24p ensure capture of diverse membrane proteins into transport vesicles. Cell 114, 497-509. 10.1016/s0092-8674(03)00609-3.
Mizzen, L., Hilton, A., Cheley, S., and Anderson, R. (1985). Attenuation of murine coronavirus infection by ammonium chloride. Virology 142, 378-388. 10.1016/0042 6822(85)90345-9.
Mykytyn, A. Z., Breugem, T. I., Riesebosch, S., Schipper, D., van den Doel, P. B., Rottier, R. J., Lamers, M. M., and Haagmans, B. L. (2021). SARS-CoV-2 entry into human airway organoids is serine protease-mediated and facilitated by the multibasic cleavage site. Elife 10. 10.7554/eLife.64508.
Newstead, S., and Barr, F. (2020). Molecular basis for KDEL-mediated retrieval of escaped ER-resident proteins—SWEET talking the COPs. J Cell Sci 133. 10.1242/jcs.250100.
Ogando, N. S., Dalebout, T. J., Zevenhoven-Dobbe, J. C., Limpens, R., van der Meer, Y., Caly, L., Druce, J., de Vries, J. J. C., Kikkert, M., Barcena, M., et al. (2020). SARS-coronavirus-2 replication in Vero E6 cells: replication kinetics, rapid adaptation and cytopathology. J Gen Virol 101, 925-940. 10.1099/jgv.0.001453.
Ohno, H., Stewart, J., Fournier, M. C., Bosshart, H., Rhee, I., Miyatake, S., Saito, T., Gallusser, A., Kirchhausen, T., and Bonifacino, J. S. (1995). Interaction of tyrosine-based sorting signals with clathrin-associated proteins. Science 269, 1872-1875. 10.1126/science.7569928.
Ozono, S., Zhang, Y., Ode, H., Sano, K., Tan, T. S., Imai, K., Miyoshi, K., Kishigami, S., Ueno, T., Iwatani, Y., et al. (2021). SARS-CoV-2 D614G spike mutation increases entry efficiency with enhanced ACE2-binding affinity. Nat Commun 12, 848. 10.1038/s41467-021-21118-2.
Pallesen, J., Wang, N., Corbett, K. S., Wrapp, D., Kirchdoerfer, R. N., Turner, H. L., Cottrell, C. A., Becker, M. M., Wang, L., Shi, W., et al. (2017). Immunogenicity and structures of a rationally designed prefusion MERS-CoV spike antigen. Proc Natl Acad Sci USA 114, E7348-E7357. 10.1073/pnas.1707304114.
Papa, G., Mallery, D. L., Albecka, A., Welch, L. G., Cattin-Ortola, J., Luptak, J., Paul, D., McMahon, H. T., Goodfellow, I. G., Carter, A., et al. (2021). Furin cleavage of SARS-CoV-2 Spike promotes but is not essential for infection and cell-cell fusion. PLoS Pathog 17, e1009246. 10.1371/journal.ppat.1009246.
Peacock, T. P., Goldhill, D. H., Zhou, J., Baillon, L., Frise, R., Swann, O. C., Kugathasan, R., Penn, R., Brown, J. C., Sanchez-David, R. Y., et al. (2021). The furin cleavage site in the SARS-CoV-2 spike protein is required for transmission in ferrets. Nat Microbiol. 10.1038/s41564-021-00908-w.
Pinto, D., Park, Y. J., Beltramello, M., Walls, A. C., Tortorici, M. A., Bianchi, S., Jaconi, S., Culap, K., Zatta, F., De Marco, A., et al. (2020). Cross-neutralization of SARS-CoV-2 by a human monoclonal SARS-CoV antibody. Nature 583, 290-295. 10.1038/s41586-020 2349-y.
Pislar, A., Mitrovic, A., Sabotic, J., Pecar Fonovic, U., Perisic Nanut, M., Jakos, T., Senjor, E., and Kos, J. (2020). The role of cysteine peptidases in coronavirus cell entry and replication: The therapeutic potential of cathepsin inhibitors. PLoS Pathog 16, e1009013. 10.1371/journal.ppat.1009013.
Poland, G. A., Ovsyannikova, I. G., and Kennedy, R. B. (2020). SARS-CoV-2 immunity: review and applications to phase 3 vaccine candidates. Lancet. 10.1016/S0140 6736(20)32137-1.
Pu, J., Keren-Kaplan, T., and Bonifacino, J. S. (2017). A Ragulator-BORC interaction controls lysosome positioning in response to amino acid availability. J Cell Biol 216, 4183-4197. 10.1083/jcb.201703094.
Ruch, T. R., and Machamer, C. E. (2012). The coronavirus E protein: assembly and beyond. Viruses 4, 363-382. 10.3390/v4030363.
Sasaki, M., Uemura, K., Sato, A., Toba, S., Sanaki, T., Maenaka, K., Hall, W. W., Orba, Y., and Sawa, H. (2021). SARS-CoV-2 variants with mutations at the S1/S2 cleavage site are generated in vitro during propagation in TMPRSS2-deficient cells. PLoS Pathog 17, e1009233. 10.1371/journal.ppat.1009233.
Sevier, C. S., Weisz, O. A., Davis, M., and Machamer, C. E. (2000). Efficient export of the vesicular stomatitis virus G protein from the endoplasmic reticulum requires a signal in the cytoplasmic tail that includes both tyrosine-based and di-acidic motifs. Mol Biol Cell 11, 13-22. 10.1091/mbc.11.1.13.
Simmons, G., Gosalia, D. N., Rennekamp, A. J., Reeves, J. D., Diamond, S. L., and Bates, P. (2005). Inhibitors of cathepsin L prevent severe acute respiratory syndrome coronavirus entry. Proc Natl Acad Sci USA 102, 11876-11881. 10.1073/pnas.0505577102.
Simmons, G., Reeves, J. D., Rennekamp, A. J., Amberg, S. M., Piefer, A. J., and Bates, P. (2004). Characterization of severe acute respiratory syndrome-associated coronavirus (SARS-CoV) spike glycoprotein-mediated viral entry. Proc Natl Acad Sci USA 101, 42404245. 10.1073/pnas.0306446101.
Sun, Z., and Brodsky, J. L. (2019). Protein quality control in the secretory pathway. J Cell Biol 218, 3171-3187. 10.1083/jcb.201906047.
Taylor, P. C., Adams, A. C., Hufford, M. M., de la Torre, I., Winthrop, K., and Gottlieb, R. L. (2021). Neutralizing monoclonal antibodies for treatment of COVID-19. Nat Rev Immunol 21, 382-393. 10.1038/s41577-021-00542-x.
Terajima, M., Cruz, J., Co, M. D., Lee, J. H., Kaur, K., Wrammert, J., Wilson, P. C., and Ennis, F. A. (2011). Complement-dependent lysis of influenza a virus-infected cells by broadly cross-reactive human monoclonal antibodies. J Virol 85, 13463-13467. 10.1128/JVI.05193-11.
Tso, F. Y., Lidenge, S. J., Poppe, L. K., Pena, P. B., Privatt, S. R., Bennett, S. J., Ngowi, J. R., Mwaiselage, J., Belshan, M., Siedlik, J. A., et al. (2021). Presence of antibody-dependent cellular cytotoxicity (ADCC) against SARS-CoV-2 in COVID-19 plasma. PLoS One 16, e0247640. 10.1371/journal.pone.0247640.
Ujike, M., and Taguchi, F. (2015). Incorporation of spike and membrane glycoproteins into coronavirus virions. Viruses 7, 1700-1725. 10.3390/v7041700.
Vanderven, H. A., Jegaskanda, S., Wheatley, A. K., and Kent, S. J. (2017). Antibody-dependent cellular cytotoxicity and influenza virus. Curr Opin Virol 22, 89-96. 10.1016/j.coviro.2016.12.002.
Volz, E., Hill, V., McCrone, J. T., Price, A., Jorgensen, D., O'Toole, A., Southgate, J., Johnson, R., Jackson, B., Nascimento, F. F., et al. (2020). Evaluating the Effects of SARS-CoV-2 Spike Mutation D614G on Transmissibility and Pathogenicity. Cell. 10.1016/j.cell.2020.11.020.
Walls, A. C., Park, Y. J., Tortorici, M. A., Wall, A., McGuire, A. T., and Veesler, D. (2020). Structure, Function, and Antigenicity of the SARS-CoV-2 Spike Glycoprotein. Cell 181, 281-292 e286. 10.1016/j.cell.2020.02.058.
Walsh, E. E., Frenck, R. W., Jr., Falsey, A. R., Kitchin, N., Absalon, J., Gurtman, A., Lockhart, S., Neuzil, K., Mulligan, M. J., Bailey, R., et al. (2020). Safety and Immunogenicity of Two RNA-Based Covid-19 Vaccine Candidates. N Engl J Med. 10.1056/NEJMoa2027906.
Wang, R., Simoneau, C. R., Kulsuptrakul, J., Bouhaddou, M., Travisano, K. A., Hayashi, J. M., Carlson-Stevermer, J., Zengel, J. R., Richards, C. M., Fozouni, P., et al. (2021). Genetic Screens Identify Host Factors for SARS-CoV-2 and Common Cold Coronaviruses. Cell 184, 106-119 e114. 10.1016/j.cell.2020.12.004.
Wang, Y., Grunewald, M., and Perlman, S. (2020). Coronaviruses: An Updated Overview of Their Replication and Pathogenesis. Methods Mol Biol 2203, 1-29. 10.1007/978-1 0716-0900-2_1.
Wei, J., Alfajaro, M. M., DeWeirdt, P. C., Hanna, R. E., Lu-Culligan, W. J., Cai, W. L., Strine, M. S., Zhang, S. M., Graziano, V. R., Schmitz, C. O., et al. (2021). Genome-wide CRISPR Screens Reveal Host Factors Critical for SARS-CoV-2 Infection. Cell 184, 76-91 e13. 10.1016/j.cell.2020.10.028.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263. 10.1126/science.abb2507.
Ye, L., Bu, Z., Vzorov, A., Taylor, D., Compans, R. W., and Yang, C. (2004). Surface stability and immunogenicity of the human immunodeficiency virus envelope glycoprotein: role of the cytoplasmic domain. J Virol 78, 13409-13419. 10.1128/JVI.78.24.13409-13419.2004.
Yurkovetskiy, L., Wang, X., Pascal, K. E., Tomkins-Tinch, C., Nyalile, T. P., Wang, Y., Baum, A., Diehl, W. E., Dauphin, A., Carbone, C., et al. (2020). Structural and Functional Analysis of the D614G SARS-CoV-2 Spike Protein Variant. Cell 183, 739-751 e738. 10.1016/j.cell.2020.09.032.
Zhang, J., Cai, Y., Xiao, T., Lu, J., Peng, H., Sterling, S. M., Walsh, R. M., Jr., Rits-Volloch, S., Zhu, H., Woosley, A. N., et al. (2021). Structural impact on SARS-CoV-2 spike protein by D614G substitution. Science 372, 525-530. 10.1126/science.abf2303.
Zhang, L., Jackson, C. B., Mou, H., Ojha, A., Peng, H., Quinlan, B. D., Rangarajan, E. S., Pan, A., Vanderheiden, A., Suthar, M. S., et al. (2020). SARS-CoV-2 spike-protein D614G mutation increases virion spike density and infectivity. Nat Commun 11, 6013. 10.1038/s41467-020-19808-4.
Zheng, Y., Shang, J., Yang, Y., Liu, C., Wan, Y., Geng, Q., Wang, M., Baric, R., and Li, F. (2018). Lysosomal Proteases Are a Determinant of Coronavirus Tropism. J Virol 92. 10.1128/JVI.01504-18.
Zhou, B., Thao, T. T. N., Hoffmann, D., Taddeo, A., Ebert, N., Labroussaa, F., Pohlmann, A., King, J., Steiner, S., Kelly, J. N., et al. (2021). SARS-CoV-2 spike D614G change enhances replication and transmission. Nature 592, 122-127. 10.1038/s41586-021-03361-1.
Zhou, H., Chen, X., Hu, T., Li, J., Song, H., Liu, Y., Wang, P., Liu, D., Yang, J., Holmes, E. C., et al. (2020a). A Novel Bat Coronavirus Closely Related to SARS-CoV-2 Contains Natural Insertions at the S1/S2 Cleavage Site of the Spike Protein. Curr Biol 30, 2196-2203 e2193. 10.1016/j.cub.2020.05.023.
Zhou, P., Yang, X. L., Wang, X. G., Hu, B., Zhang, L., Zhang, W., Si, H. R., Zhu, Y., Li, B., Huang, C. L., et al. (2020b). A pneumonia outbreak associated with a new coronavirus of probable bat origin. Nature 579, 270-273. 10.1038/s41586-020-2012-7.
Barlowe, C. K., and Miller, E. A. (2013). Secretory protein biogenesis and traffic in the early secretory pathway. Genetics 193, 383-410. 10.1534/genetics.112.142810.
Braulke, T., and Bonifacino, J. S. (2009). Sorting of lysosomal proteins. Biochim Biophys Acta 1793, 605-614. 10.1016/j.bbamcr.2008.10.016.
Caras, I. W., and Weddell, G. N. (1989). Signal peptide for protein secretion directing glycophospholipid membrane anchor attachment. Science 243, 1196-1198. 10.1126/science.2466338.
McBride, C. E., Li, J., and Machamer, C. E. (2007). The cytoplasmic tail of the severe acute respiratory syndrome coronavirus spike protein contains a novel endoplasmic reticulum retrieval signal that binds COPI and promotes interaction with membrane protein. J Virol 81, 2418-2428. 10.1128/JVI.02146-06.
Miller, E. A., Beilharz, T. H., Malkus, P. N., Lee, M. C., Hamamoto, S., Orci, L., and Schekman, R. (2003). Multiple cargo binding sites on the COPII subunit Sec24p ensure capture of diverse membrane proteins into transport vesicles. Cell 114, 497-509. 10.1016/s0092-8674(03)00609-3.
Muniz, M., and Riezman, H. (2016). Trafficking of glycosylphosphatidylinositol anchored proteins from the endoplasmic reticulum to the cell surface. J Lipid Res 57, 352-360. 10.1194/jlr.R062760.
Ohno, H., Stewart, J., Fournier, M. C., Bosshart, H., Rhee, I., Miyatake, S., Saito, T., Gallusser, A., Kirchhausen, T., and Bonifacino, J. S. (1995). Interaction of tyrosine-based sorting signals with clathrin-associated proteins. Science 269, 1872-1875. 10.1126/science.7569928.
Pallesen, J., Wang, N., Corbett, K. S., Wrapp, D., Kirchdoerfer, R. N., Turner, H. L., Cottrell, C. A., Becker, M. M., Wang, L., Shi, W., et al. (2017). Immunogenicity and structures of a rationally designed prefusion MERS-CoV spike antigen. Proc Natl Acad Sci USA 114, E7348-E7357. 10.1073/pnas.1707304114.
Sevier, C. S., Weisz, O. A., Davis, M., and Machamer, C. E. (2000). Efficient export of the vesicular stomatitis virus G protein from the endoplasmic reticulum requires a signal in the cytoplasmic tail that includes both tyrosine-based and di-acidic motifs. Mol Biol Cell 11, 13-22. 10.1091/mbc.11.1.13.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263. 10.1126/science.abb2507.

Although the invention has been described with reference to the above examples, it will be understood that modifications and variations are encompassed within the spirit and scope of the invention. Accordingly, the invention is limited only by the following claims.

1. An isolated non-naturally occurring cell-surface protein comprising an extracellular domain, a transmembrane domain and a carboxy-terminal cytoplasmic domain, wherein the carboxy-terminal cytoplasmic domain comprises a deletion or addition of a heterologous sequence for improved cell-surface expression of the protein relative to the protein prior to the deletion or addition of the sequence.

2. The protein of claim 1, wherein the protein is a therapeutic protein or an antigenic protein.

3. The protein of claim 1, wherein the carboxy-terminal cytoplasmic domain comprises an endoplasmic reticulum (ER) export signal.

4. The protein of claim 1, with the proviso that the cytoplasmic domain does not have an HDEL (SEQ ID NO:1) or a KDEL (SEQ ID NO:2) sequence.

5. The protein of claim 1, comprising the sequence KLKHTKKRQIYTDIEMNRLGK (SEQ ID NO:3) at the carboxy-terminus of the protein.

6. The protein of claim 1, comprising the sequence Tyr19-Thr20-Asp21-Ile22-Glu23-Met24 (SEQ ID NO:4) of vesicular stomatitis virus glycoprotein (VSV G) tail at the carboxy-terminus of the protein.

7. The protein of claim 1, wherein the protein is an antigenic protein from a eukaryote, a prokaryote or a virus.

8. The protein of claim 7, wherein the virus is a coronavirus.

9. The protein of claim 8, wherein the coronavirus is SARS-CoV-2.

10. The protein of claim 9, wherein the antigenic protein is spike protein.

11. The protein of claim 10, wherein all or part of the sequence of the cytoplasmic domain of the SARS-CoV-2 spike protein is removed.

12. The protein of claim 11, wherein the sequence KFDEDDSEPVLKGVKLHYT_{COOH} (SEQ ID NO:5) in the cytoplasmic domain of the spike protein is removed.

13. The protein of claim 1, wherein the SARS-CoV-2 spike protein is the Wuhan-1 strain SARS-CoV-2 spike protein; a furin-blocked, trimer-stabilized form of the Wuhan-1 strain SARS-CoV-2 spike protein; the Wuhan-1 strain SARS-CoV-2 spike protein with an amino acid change of D614G; the Wuhan-1 strain SARS-CoV-2 spike protein with di-proline substitutions of 986KV987-to-986PP987 (S-2P); the Wuhan-1 strain SARS-CoV-2 spike protein with cleavage site mutations of 682RRAR685-to-682GSAG685, or equivalent (S-CSM); or the Wuhan-1 strain SARS-CoV-2 spike protein with both S-2P and S-CSM mutations.

14. An isolated nucleic acid sequence encoding the protein of claim 1.

15-17. (canceled)

18. An isolated cell comprising the nucleic acid sequence of any of claim 14.

19. The cell of claim 18, wherein the cell is a mammalian cell.

20-27. (canceled)

28. A pharmaceutical composition comprising the nucleic acid sequence of claim 14 and a physiologically acceptable excipient and/or adjuvant.

29-30. (canceled)

31. The pharmaceutical composition of claim 28, wherein the EV is an exosome or a microvesicle.

32-38. (canceled)

39. A method comprising administering into an animal a pharmaceutical composition of claim 28.

40-45. (canceled)
